(19) United States
(12) Patent Application Publication
(10) Pub. No.: US 2010/0221749 A1
(43) Pub. Date: Sep. 2, 2010

(54) THREE-FUNCTIONAL PSEUDO-PEPTIDIC REAGENT, AND USES AND APPLICATIONS THEREOF

(75) Inventors: Guillaume Clave, Rouen (FR); Pierre-Yves Renard, Paris (FR); Anthony Romieu, Rouen (FR); Herve Volland, Orsay (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: UNIVERSITE DE ROUEN

(21) Appl. No.: 12/668,518

(22) PCT Filed: Jul. 10, 2008
(86) PCT No.: PCT/FR08/01007

§ 371 Date: May 13, 2010

(30) Foreign Application Priority Data
Jul. 11, 2007 (FR) 0705018

(51) Int. Cl. G01N 33/573; C07D 207/452; C07D 207/46; C07C 321/04; C07K 17/02; C07K 16/00; C07H 21/00; C07H 3/00

(52) U.S. Cl. 435/7.4; 548/521; 548/542; 560/10; 530/350; 530/387.3; 536/23.1; 536/123.1

(57) ABSTRACT

The invention relates to a three-functional pseudo-peptidic reagent, to the various uses thereof, in particular for preparing bioluminescent reagents or optionally luminescent bio-conjugates, to the use of said reagents and bio-conjugates for functionalising solid substrates, and to the use of solid substrates thus functionalised in the detection of molecules of interest.

The present invention relates to a pseudopeptide trifunctional reagent, to the various uses thereof, especially for the preparation of luminescent reagents or of optionally luminescent bioconjugates, to the use of these reagents in bioconjugates for the functionalization of solid supports, and also to the use of the thus functionalized solid supports for the detection of molecules of interest.

Many applications that bring into play bioconjugates derived from biopolymers (nucleic acids, proteins, polysaccharides, etc.) involve being able to covalently attach (reversible or irreversible attachment) these bioconjugates to a second molecular architecture (biopolymer, solid support, etc.) and to detect them and/or quantify them with precision (optical detection, radioactive detection, etc.). Thus, it is essential to have tools that make it possible to effectively combine a biopolymer with other (macro)molecules without too greatly impairing the properties of each of the partners involved in the resulting and targeted molecular architecture.

For this purpose, many small bifunctional molecules (better known by the expression “bifunctional cross-linking reagents”) have been developed. A large number is sold by the company Pierce. However, it is interesting to note that several academic groups continue to work on the development of novel bifunctional reagents that are more and more sophisticated and that make it possible to produce more and more complex bioconjugates.

As mentioned previously, it is sometimes essential to have not two but three orthogonal functions (i.e. each function is capable of reacting selectively with a targeted partner). Thus, the development of trifunctional reagents is called for.

In this way, the use of various types of trifunctional reagents has already been described, for example in the articles by Alley, S. C. et al., J. Am. Chem. Soc., 2000, 122, 6126-6127 and by Sinz, A. et al., J. Am. Soc. Mass Spectrom., 2005, 16, 1921-1931, among which trifunctional reagents there is, in particular, the sulfo-SBED available from the company Pierce (Rockford, Ill., USA), that is to say sulfosuccinimidyl-2-[6-(biotinamido)-2-(p-azidobenzimidazo)hexanoamido]ethyl-1,3′-dithiopropionate, for the study of protein/protein interactions. This trifunctional reagent is composed of a reactive amine and of a photoreactive site. Sulfo-SBED has the following structure:

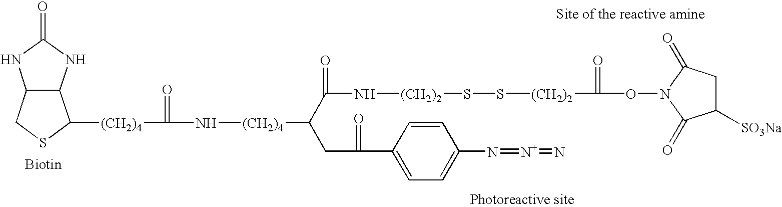

However, sulfo-SBED is not completely satisfactory insofar as it is not possible to adjust the hydrophobic/hydrophilic nature thereof and above all the third functional unit (i.e. the biotin) permits only a strong (quasi-covalent) interaction with partners previously conjugated to avidin (glyco protein) or streptavidin, which greatly limits the use thereof.

Trifunctional reagents have also already been described, especially in international application WO 00/02050, that make it possible to prepare bioconjugates constituted of a trifunctional central unit chosen from triaminobenzene, tricarboxybenzene, dicarboxyaniline and diaminobenzoic acid, to which are attached, via three different linkers, an affinity ligand, a group that is reactive with respect to a biomolecule and an effector agent. This reagent has however the drawback of possessing a trifunctional central unit constituted of at least two identical chemical functional groups (i.e. carboxylic acid or amine functional groups) which restricts the choice of complementary functional groups borne by the linkers which may have a limiting effect.

In other applications, especially for the detection of analytes by heterogeneous immunofluorescence, it has already been proposed to use trifunctional reagents (tripods) comprising three different functional poles, namely a luminescent group (L), a molecule (B) chosen from the analyte to be detected, an analog or a fragment of this and finally a functional group that ensures the attachment of said trifunctional reagent to the surface of a solid support (see, in particular, the French patent application published under the number FR 2 847 984 and the corresponding article by Volland, H. et al., Anal. Chem., 2005, 77, 1986-1904). These reagents cannot however be used in all types of application.

The development of novel, ever more precise technologies requires the creation and use of ever more sophisticated bioconjugates that it is not easy (or is even impossible) to achieve with the bifunctional or trifunctional reagents currently available on the market.

It is therefore in order to overcome all these drawbacks and to provide, in particular, a novel family of trifunctional reagents in which it is possible to very widely vary the nature of each functional group in order to be able to effectively combine a biopolymer with other (macro)molecules without too greatly impairing the properties of each of the partners involved in the resulting and targeted molecular architecture that the inventors have developed what is the subject of the present invention. More specifically, the inventors have made it their aim to provide a trifunctional reagent that makes it possible to covalently bond (reversible or irreversible attachment) a bioconjugate to a second molecular architecture such as a biopolymer or a solid support.

A first subject of the present invention is therefore a pseudopeptide trifunctional reagent, characterized by the fact that it comprises at least the following three reactive structural units A, B and C:

(a) a unit A constituted of a hydrophilic chain of pseudo-polyethylene glycol (pseudo-PEG chain) interrupted by at least one amide functional group and having two ends E1 and E2, said end E1 being free and comprising a terminal unit chosen from amino group (—NH₂), activated carbamates and activated esters, and said end E2 comprising a terminal carbon atom bearing a carbonyl functional group, said carbon atom being engaged in an amide (—C(O)—NH—) functional group formed with the nitrogen atom of an α-amine functional group borne by the unit B;

(b) a unit B constituted of an amino acid chosen from the α-amino acids of the L or D series and racemic mixtures thereof, said amino acid having on its side chain at least one oxyamine functional group protected by a protecting group or at least one masked aldehydic functional group; and

(c) a unit C constituted of an amino acid chosen from the α-amino acids of the L or D series and racemic mixtures thereof, said amino acid having on its side chain at least one thiol, maleimide, iodoacetyl, azide, true alkyne, phosphane or cyclooctyne unit;

said units B and C being linked together via an amide functional group formed between the carbon atom bearing the carbonyl functional group of the α-amino acid constituting the unit B and the nitrogen atom of the α-amine functional group of the α-amino acid constituting the unit C.

The main originality of these structures lies in the combination of (natural or modified) amino acids, protected on their side chains by selected protecting groups, and of a functionalized pseudo-PEG chain, the length of which can be easily adjusted. Even though the presence of several (protected or unprotected) chemical functional groups inevitably leads to problems during the preparation of these reagents (incompatibilities between functional groups, premature deprotection and/or degradation of certain protecting groups, etc.), the structure, segmented into three separate structural units (prepared independently of one another then coupled together during the final steps of producing the trifunctional reagent) allows a highly convergent synthesis strategy which limits as much as possible the simultaneous presence of chemical units that are incompatible with one another. Furthermore, a great structural diversity (adjustment of the geometry, of the length, of the physicochemical properties and the solubility in particular, etc.) is accessible by modifying only one of the three structural units of the trifunctional reagent.

According to the invention, the expression “pseudo”-PEG chain is understood to mean a chain having great structural similarities with the PEG chain:

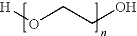

but which differs via the presence of one or more functional groups (ester, amide, carbamate, urea, etc.) within it.

According to the invention, the term “true” alkyne is understood to mean an alkyne whose triple bond is monosubstituted by an R group:

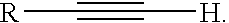

The three reactive structural units constituting the trifunctional reagents in accordance with the present invention make it possible to carry out completely chemoselective reactions under mild conditions (in particular in the aqueous media in which the biopolymers are soluble).

The activated carbamate or activated ester unit which may be present at the free end E1 of the pseudo-PEG chain constituting the unit A is reactive with respect to compounds possessing a complementary reactive unit such as an amine functional group (generally an aliphatic primary amine). Furthermore, the primary amine functional group which may alternatively be present at the free end E1 of the pseudo-PEG chain constituting the unit A is reactive with respect to compounds possessing a complementary unit such as an activated carbamate or ester. This end E1 enables, in particular, the attachment of biological macromolecules which comprise, naturally or otherwise, said complementary reactive functional groups (antibodies, nucleic acids or analogs, polysaccharides, proteins, peptides, radionuclides, toxins, enzyme inhibitors, haptens, etc.).

The group of the unit B, after optional activation, that is to say after deprotection in the case of the oxyamine functional group or after demasking in the case of the aldehydic functional group, under mild conditions that are compatible with the stability of the various partners involved, is reactive with respect to compounds or materials possessing one or more carbonyl-containing (aldehyde, etc.) or alternatively amino groups. Among such compounds, mention may especially be made of macromolecules such as antibodies, nucleic acids or analogs, liposomes, polysaccharides, proteins, peptides, active principles, radionuclides, toxins, fluorophores, enzyme inhibitors, haptens, etc.

The thiol unit defined as a possible side substituent of the α-amino acid constituting the unit C is reactive with respect to compounds or materials comprising a maleimide or iodoacetyl unit. The maleimide and iodoacetyl units defined as possible side substituents of the α-amino acid constituting the unit C are reactive with respect to compounds or materials possessing a thiol functional group or a cysteine after their optional deprotection. Finally, the azide unit defined as a possible side substituent of the α-amino acid constituting the unit C is reactive with respect to compounds or materials possessing a true alkyne, phosphane or cyclooctyne unit and alternatively the true alkyne, phosphane or cyclooctyne units defined as possible side substituents of the α-amino acid constituting the unit C are reactive with respect to compounds or materials possessing an azide unit. This reaction site may especially be used for the attachment of a fluorophore group bearing a complementary functional group that is reactive with respect to thiol, maleimide, iodoacetyl, azide, true alkyne, phosphane or cyclooctyne units present on the unit C.

According to one particular embodiment form of the present invention, the pseudo-PEG chain of the unit A is chosen from the chains of formula (A-I) below:

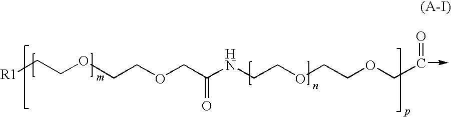

in which:
R₁ represents a primary amine, an activated carbamate unit or an activated ester unit,
m and n, which are identical or different, are integers between 2 and 10 inclusive,
p is an integer between 1 and 10 inclusive,
the arrow represents the covalent bond connecting the amide functional group of the end E2 of the pseudo-PEG chain to the unit B or C.

According to one preferred embodiment form of the invention, the pseudo-PEG chain is chosen from the pseudo-PEG chains of formula (A-I) above in which m=n=2 and p=1.

Among the activated carbamate groups which may be present at the end E1 of the pseudo-PEG chain of the unit A, mention may in particular be made of N-hydroxy-succinimidyl carbamate, sulfo-N-hydroxysuccinimidyl carbamate, N-hydroxyphthalimidyl carbamate, N-hydroxypiperidyl carbamate, p-nitrophenyl carbamate and pentafluorphenyl carbamate; N-hydroxysuccinimidyl carbamate being very particularly preferred.

Among the activated ester groups which may be present at the end E1 of the pseudo-PEG chain of the unit A, mention may in particular be made of N-hydroxy-succinimidyl ester, sulfo-N-hydroxysuccinimidyl ester, cyanomethyl ester, N-hydroxyphthalimidyl ester, N-hydroxypiperidyl ester, p-nitrophenyl ester, pentafluorophenyl ester, benzotriazole esters, hydroxybenzotriazole esters and hydroxyazabenzotriazole esters; the N-hydroxysuccinimidyl ester being particularly preferred.

Among the α-amino acids that can be used for the unit B, mention may especially be made of lysine, homolysine, ornithine, 2,4-diaminobutanoic acid (DABA) and 2,3-diaminopropanoic acid.

Among such α-amino acids, lysine is particularly preferred.

Among the α-amino acids that can be used for the unit C, mention may especially be made of lysine, cysteine, homolysine, ornithine, 2,4-diaminobutanoic acid (DABA), 2,3-diaminopropanoic acid, aminomercaptoacetic acid, homocysteine, 5-mercaptonorvaline, 6-mercapto-norleucine, 2-amino-7-mercaptoheptanoic acid and 2-amino-8-mercaptooctanoic acid.

Among such amino acids, lysine and cysteine are particularly preferred.

The protecting groups for the oxyamine functional group of the side chain of the α-amino acid constituting the unit B are preferably chosen from the protecting groups that are labile under mild conditions such as 9-fluorenylmethoxycarbonyl (Fmoc), benzyloxycarbonyl (Z), allyloxycarbonyl (Alloc), trichloroethoxycarbonyl (Troc), trimethylsilylethoxycarbonyl (Teoc), pyridyl-dithioethoxycarbonyl (Pydec), 2-(2-nitrophenyl)propyl-oxycarbonyl (NPPOC), azomethyloxycarbonyl (Azoc), 2-(trimethylsilyl)ethanesulfonyl (Ses) and phthalamide.

Among such protecting groups, the Fmoc and phthalamide groups are particularly preferred.

Within the meaning of the present invention, the expression “masked aldehydic functional group” is understood to mean any aldehyde or ketone functional group included in an organic molecule chosen from serine or any organic molecule containing the 1,2-diol (—CH(OH)—CH(OH)—), 1,2-aminoalcohol or 1,2-hydroxythiol unit. Among such organic molecules, mention may especially be made of tartaric acid, glyceric acid, 2,3-dihydroxypropanoic acid, 3,4-dihydroxybutanoic acid, 4,5-dihydroxypentanoic acid and 3-amino-2-hydroxypropanoic acid.

Within the meaning of the present invention, the expression “mild conditions” is understood to mean the use of reagents for deprotecting the oxyamine functional group or demasking the aldehydic functional group that operate at ambient temperature and at neutral pH or in a pH range between around 5 and 9 inclusive.

As a deprotecting or demasking reagent, mention may especially be made of periodic acid (HIO₄), metaperiodates such as sodium metaperiodate (NaIO₄) and potassium metaperiodate (KIO₄), and also tetraalkylammonium periodates.

Among the trifunctional reagents in accordance with the invention, mention may very particularly be made of those which are chosen from the compounds of formula (I) below:

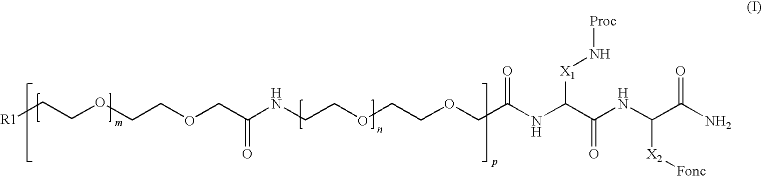

in which:
R₁, m, n and p have the same meanings as those defined above for the unit of formula (A-I),
X₁ and X₂, which are identical or different, represent, together with the carbon atom to which they are attached, the hydrocarbon-based chain of an α-amino acid,
Proc is a protecting group chosen from 9-fluorenylmethoxycarbonyl (Fmoc), benzyloxycarbonyl (Z), allyloxycarbonyl (Alloc), trichloroethoxycarbonyl (Troc), trimethylsilylethoxycarbonyl (Teoc), pyridyl-dithioethoxycarbonyl (Pydec), 2-(2-nitrophenyl)propyl-oxycarbonyl (NPPOC), azomethyloxycarbonyl (Azoc), 2-(trimethylsilyl)ethanesulfonyl (Ses) and phthalimide;
Fonc is a thiol, maleimide, iodoacetyl, azide, true alkyne, phosphane or cyclooctyne unit.

According to one particularly preferred embodiment form of the invention, X₁ is an n-butyl chain.

According to another particularly preferred embodiment form of the invention, X₂ is an ethyl or n-butyl chain.

According to one particularly preferred embodiment form of the invention, the compounds of formula (I) above are chosen from the compounds of formula (I-1) to (I-6) below:

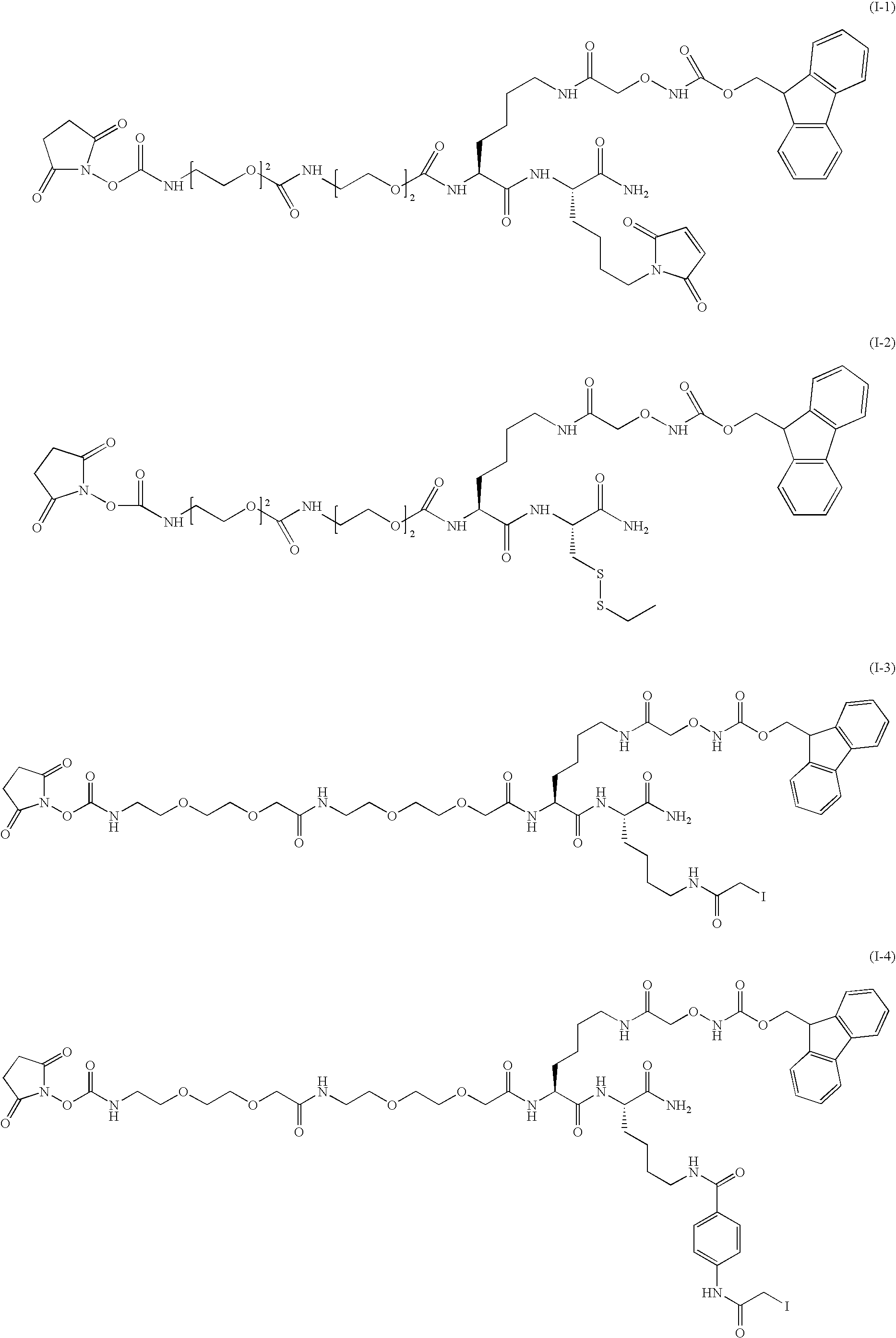

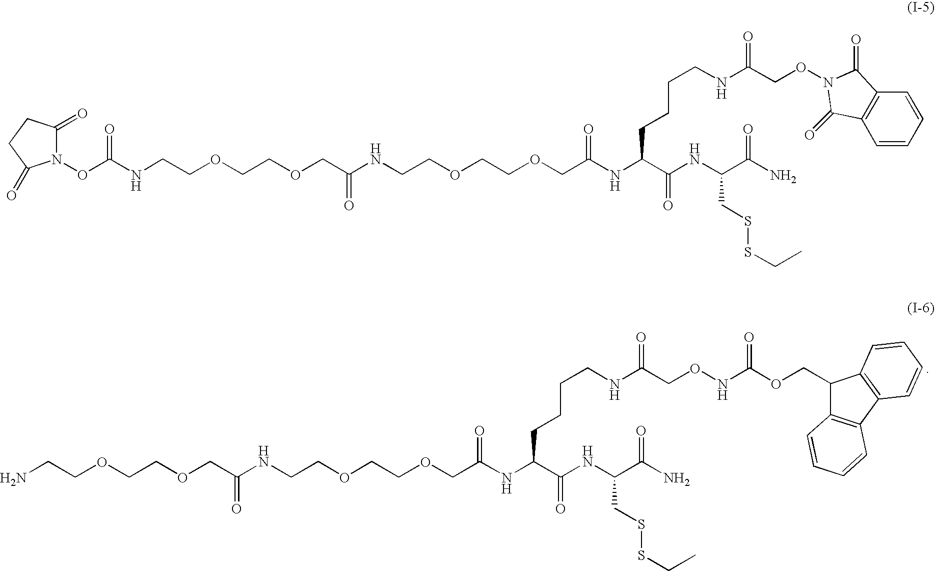

The trifunctional reagents in accordance with the invention may be prepared according to convergent synthesis methods according to which each of the units A, B and C is prepared individually, these then being assembled in order to lead to the expected trifunctional reagent. These convergent synthesis methods use conventional reactions well known to a person skilled in the art, the details of which are given in the synthesis examples that illustrate the present application.

By virtue of their trifunctionality, the reagents in accordance with the present invention and as described above may have multiple uses and applications.

The trifunctional reagents in accordance with the present invention may firstly be used for the preparation of bioconjugates.

Another subject of the present invention is therefore the use of at least one trifunctional reagent as defined previously for the preparation of a bioconjugate.

Within the meaning of the present invention, the term “bioconjugate” is understood to mean any trifunctional reagent as described previously attached to which is at least one biological molecule of interest.

The attachment of the molecule or molecules of interest may be carried out at the primary amine functional group or the activated carbamate or activated ester unit present at the free end of the pseudo-PEG chain constituting the unit A which is reactive with respect to biological molecules possessing an acid (or carbamate) functional group or a reactive amine functional group (generally an aliphatic primary amine).

The attachment of the biological molecule of interest may also be carried out at the unit B, after deprotection of the oxyamine functional group or demasking of the aldehydic functional group, which then become reactive with respect to biological molecules that possess one or more carbonyl-containing (aldehyde, etc.), or respectively oxyamine, groups.

It is thus possible to attach one or two biological molecules, which are identical or different, via the trifunctional reagent in accordance with the present invention.

Among the biological molecules which may be attached to the trifunctional reagents in accordance with the present invention, mention may especially be made of antibodies, molecules of nucleic acids and their analogs, polysaccharides, proteins, peptides, radionuclides, toxins, enzyme inhibitors, haptens, etc.

Another subject of the invention is therefore a bioconjugate, characterized by the fact that it consists of a trifunctional reagent as defined previously, in which the primary amine, activated carbamate or activated ester unit present at the free end of the pseudo-PEG chain constituting the unit A and/or the oxyamine or aldehydic functional group borne by the unit B after its deprotection, respectively its demasking, is (are) functionalized by a biological molecule of interest.

According to the invention, it is therefore possible to have the following three types of bioconjugates:

i) a bioconjugate constituted by a trifunctional reagent in which only the amine functional group or only the activated carbamate or activated ester unit present at the free end of the pseudo-PEG chain constituting the unit A is functionalized by a biological molecule,
ii) a bioconjugate constituted by a trifunctional reagent in which only the deprotected oxyamine or demasked aldehydic functional group of the unit B is functionalized by a biological molecule, and
iii) a bioconjugate comprising two biological molecules, constituted by a trifunctional reagent in which the primary amine functional group or activated carbamate or activated ester unit present at the free end of the pseudo-PEG chain constituting the unit A and the deprotected oxyamine or demasked aldehydic functional group of the unit B are each functionalized by a biological molecule; in the latter case the bioconjugate comprises two biological molecules which may be identical to or different from one another. It may in particular be a question of two biological molecules that are identical but that are modified by two different markers.

The preparation of these bioconjugates may be carried out conventionally by reacting a trifunctional reagent in accordance with the invention with the biological molecule or molecules of interest to be attached, while using the well-known methods of the prior art for reacting, for example:
an activated carbamate or activated ester with an amine functional group (Hermanson G. T., Bioconjugate Techniques, 1996, Academic Press, Inc.) or
an oxyamine functional group with a carbonyl-containing group (Sing, Y. et al., Org. Biomol. Chem., 2006, 4, 1413-1419 or Zatsepin, T. S. et al., Bioconjugate Chem., 2005, 16, 471-489.

The trifunctional reagents in accordance with the present invention may also be used for the preparation of luminescent reagents, in particular fluorescent reagents.

Another subject of the present invention is therefore the use of at least one trifunctional reagent as defined previously, for the preparation of a luminescent reagent, in particular fluorescent reagents.

In this case, the grafting of a luminescent group (L) may be carried out:

i) by reacting the thiol, maleimide, iodoacetyl, azide, true alkyne, phosphane or cyclooctyne unit borne by the unit C with a complementary maleimide or iodoacetyl functional group (if the unit C comprises a thiol functional group), a thiol functional group (if the unit C comprises a maleimide or iodoacetyl unit) or a true alkyne, phosphate or cyclooctyne functional group (if the unit C comprises an azide functional group), or else an azide functional group (if the unit C comprises a true alkyne, phosphane or cyclooctyne functional group), said complementary functional group being borne, naturally or otherwise, by said luminescent group,
and/or
ii) by reacting the deprotected oxyamine or demasked aldehydic functional group of the unit B with a luminescent group bearing a carbonyl-containing functional group such as an aldehyde or ketone functional group, or respectively with an oxyamine functional group.

In these two cases, it may optionally be necessary to previously functionalize the luminescent group to be reacted with a unit complementary to the functional group with which it is desired to react it.

The preparation of these luminescent reagents may be carried out conventionally according to the reactions known to a person skilled in the art.

It is thus possible to obtain luminescent reagents comprising:

i) a single luminescent group attached to the unit B by means of the deprotected oxyamine functional group or the demasked aldehydic functional group, or to the unit C by means of the thiol functional group or the maleimide, iodoacetyl, true alkyne, phosphane, cyclooctyne or else azide units;
ii) two luminescent groups, one being attached to the unit B by means of the deprotected oxyamine functional group or the demasked aldehydic functional group, and the other being attached to the unit C by means of the thiol functional group or the maleimide, iodoacetyl, true alkyne, phosphane, cyclooctyne or else azide units.

These luminescent trifunctional reagents constitute another subject of the invention.

The nature of the luminescent group or groups that can be used according to the invention is not critical as long as they comprise naturally, or they are functionalized by, a thiol or carbonyl-containing functional group, or else by a maleimide, iodoacetyl, true alkyne, phosphane, cyclooctyne or else azide unit.

According to the invention, the expression “luminescent group” is understood to mean any substance which, when it is excited at a given wavelength or by a given chemical compound, is capable of emitting a photon, for example a fluorophore or rare earth element.

Among the luminescent groups (including fluorophores) that can be used according to the invention, mention may in particular be made of fluorophores containing polymethine chains (i.e. polyene chain); fluorescent cyanines such as those sold under the references Cy3, Cy3.5, Cy3B, Cy5, Cy5.5 and Cy7 by the company GE Healthcare; fluorescein (sodium fluoresceinate) and derivatives thereof such as fluorescein isothiocyanate (FITC) and 6-carboxyfluorescein (6-Fam); rhodamine and derivatives thereof such as tetramethylrhodamine isothiocyanate (TRITC); the water-soluble derivatives of rhodamine in the form of ester of N-hydroxy-succinimide such as the products sold under the trade name Alexa Fluor® by the company Invitrogen such as for example the Alexa Fluor® 488, 500, 514, 532, 546, 555, 568, 594, 610-X, 633, 647, 660, 680, 700, 750 and 790 products; rhodols and derivatives thereof; derivatives of coumarin such as 7-aminocoumarin; 9-aminoacridine and 9-acridinecarboxylic acid; fluorescent dyes containing reactive amines such as the succinimidyl ester of 6-((7-amino-4-methylcoumarin-3-acetyl)amino)hexanoic acid (AMCA); the dipyrromethene boron difluorides sold under the trade names BODIPY® such as BODIPY® FR-Br₂, BODIPY® R6G, BODIPY® TMR, BODIPY® TR and BODIPY® 530/550 (excitation wavelength/emission wavelength, in nm), 558/567, 564/570, 576/589, 581/591, 630/650 and 650/665 sold by the company Bio-Rad Inc. (USA); the fluorophores derived from pyrene such as for example the dyes Cascade Blue (sold for example by the companies Trilink BioTechnologies (USA) or Invitrogen; diazo derivatives such as DABCYL®; dansyl derivatives such as EDANS®(Eurogentec, BE); eosin; erythrosine and sulforhodamine derivatives such as sulforhodamine 101 sulfonyl chloride also known under the name Texas Red.

The trifunctional reagents in accordance with the present invention may also be used for the preparation of luminescent reagents, in particular fluorescent reagents that also comprise an “acceptor compound” (Q) that accepts the luminescence from the luminescent group. In this case, such reagents constitute what is commonly known as an energy transfer cassette (or FRET cassette) that can be used in particular for DNA sequencing.

Another subject of the present invention is therefore the use of at least one trifunctional reagent as defined previously for the preparation of an energy transfer cassette.

In this case, the luminescent group will be grafted to the deprotected oxyamine or demasked aldehydic functional group of the unit B or else via the thiol functional group or the maleimide, iodoacetyl, true alkyne, phosphane, cyclooctyne or else azide units of the unit C, and the acceptor compound (Q) will be respectively grafted via the thiol functional group or the maleimide, iodoacetyl, true alkyne, phosphane, cyclooctyne or else azide units of the unit C or else to the deprotected oxyamine or demasked aldehydic functional group of the unit B.

Another subject of the invention is therefore an energy transfer cassette, characterized by the fact that it is constituted of a trifunctional reagent as defined previously, said reagent comprising a luminescent group (L) and an acceptor compound (Q) that accepts the luminescence from the luminescent group, L and Q being respectively and indifferently attached to said trifunctional reagent via the deprotected oxyamine or demasked aldehydic functional group of the unit B and the thiol functional group or the maleimide, iodoacetyl, true alkyne, phosphane, cyclooctyne or else azide units of the unit C.

In these two cases, it may optionally be necessary to previously functionalize the acceptor compound (Q) to be reacted with a unit complementary to the functional group with which it is desired to react it.

The luminescent group (L) that can be used in these energy transfer cassettes may especially be chosen from the luminescent groups (L) cited previously and that can be used for the preparation of the luminescent trifunctional reagents in accordance with the present invention.

According to the invention, the expression “acceptor compound” (Q) is understood to mean any molecule that enables the reduction or the disappearance of the luminescence from the luminescent group (L) under certain conditions. This compound, of various natures, may especially be a chemical compound (luminescent or not, such as for example fluorescent proteins), a heavy atom or a nanoparticle.

Among such acceptor compounds (Q), mention may in particular be made of the fluorescent compounds such as those mentioned above for the L groups, in particular rhodamine and derivatives thereof such as tetramethylrhodamine (TMR), Rhodamine 6G (R6G) and the dyes QSY® 7, QSY® 9 and QSY® 21 (Molecular Probes); but also non-fluorescent molecules of the family of azo dyes such as the compounds sold under the trade names Black Hole Quencher° (BHQ) such as for example BHQ-0, BHQ-1, BHQ-2 and BHQ-3 (Biosearch Technologies); gold particles such as those having a diameter of 1.5 nm sold under the trade name Nanogold Particules® (Nanoprobes); diazo dyes such as the products sold under the trade names Eclipse Dark Quencher® (Epoch Bioscience) or QSY® 35 (Molecular Probes); the commercial product ElleQuencher® (Eurogentec); malachite green and the acceptor compounds (“Quenchers”) of the family of cyanines such as the compounds sold under the trade names Cy5Q or Cy7Q by the company GE Healthcare.

According to one particularly advantageous embodiment form of these energy transfer cassettes, the luminescent group (L) and the acceptor compound (Q) are chosen from the following (L/Q) pairs: Cy3/Cy5, Cy5/Cy7, Cy5/Alexa Fluor® 750, Cy3/Cy5Q, Cy3/QSY® 7, Cy3/QSY® 9, Cy5/Cy7Q, Cy5/QSY® 21, Cy5/Cy5, Cy5.5/Cy5.5, Cy7/Cy7, R6)/Cy5, R6G/Alexa Fluor® 647, R6G/QSY® 21, Alexa Fluor® 532/Cy5, Alexa Fluor® 532/Alexa Fluor® 647, Alexa Fluor® 532/QSY® 21, Alexa Fluor® 555/Cy5, Alexa Fluor® 555/Alexa Fluor® 647, Alexa Fluor® 555/QSY® 21.

According to another particular embodiment form of the invention, the trifunctional reagents may be used for the preparation of mixed bioconjugates comprising at least one biological molecule and at least one luminescent group.

Another subject of the present invention is therefore a mixed bioconjugate, characterized by the fact that it consists of a trifunctional reagent as defined previously, attached to which are at least one biological molecule and at least one luminescent group. Moreover, these mixed bioconjugates may also comprise an acceptor compound (Q) that accepts the luminescence from the luminescent group (L).

These mixed bioconjugates are therefore chosen from the trifunctional reagents to which are attached:
i) one or two biological molecules of interest and a luminescent group; or
ii) one biological molecule of interest and two luminescent groups;
iii) one biological molecule of interest, one luminescent group, and one acceptor compound (Q) that accepts the luminescence from the luminescent group;
   said biological molecules, said luminescent groups and said acceptor compound (Q) being attached to the functional reagent at the terminal ends of the units A, B and C as described above.

The trifunctional reagents and the bioconjugates in accordance with the present invention, modified or not by a luminescent group at the unit C, and in which the oxyamine or respectively aldehydic functional group of the unit B is free (that is to say not functionalized by a biological molecule of interest, a fluorescent group or an acceptor compound) may be used for the functionalization of solid supports comprising at least one surface that possesses one or more carbonyl-containing groups, in particular one or more aldehyde or ketone functional groups or respectively one or more oxyamine functional groups.

Consequently, another subject of the present invention is a solid support, characterized by the fact that it comprises at least one surface that is functionalized, covalently, by one or more trifunctional reagents, and/or by one or more bioconjugates, said trifunctional reagents and bioconjugates optionally being modified by a luminescent group at the unit C and as defined previously.

According to the invention, the nature of the solid support is not critical as long as it comprises at least one surface that has, naturally or after chemical modification, one or more carbonyl-containing groups, in particular one or more aldehyde or ketone functional groups or respectively one or more primary amine functional groups, said groups or functional groups being capable of reacting with the deprotected oxyamine functional group or respectively with the demasked aldehydic functional group of the unit B of the trifunctional reagent or of the bioconjugate.

Among such supports, mention may especially be made of glass, plastic and metals.

According to one particular embodiment form of the invention, the solid support comprises at least one surface functionalized by at least one bioconjugate and then constitutes a biochip such as for example a nucleic acid chip, a protein chip, a polysaccharide chip or a peptide chip, or else a biosensor such as for example an immunosensor.

Such supports may be prepared according to a process that comprises the following steps:
the deprotection of the protected oxyamine functional group or respectively the demasking of the masked aldehydic functional group, present on the unit B of at least one trifunctional reagent or of at least one bioconjugate in accordance with the invention in order to obtain a trifunctional reagent or a bioconjugate bearing a deprotected oxyamine, respectively demasked aldehydic, functional group,
the formation of an oxime bond by bringing said reagent or said bioconjugate bearing the deprotected oxyamine or respectively demasked aldehydic functional group into contact with a solid support, at least one surface of which possesses one or more carbonyl-containing, respectively primary amine, groups, said oxime bond providing the covalent attachment of said reagent or of said bioconjugate to the surface of the support.

Such supports may especially be used for the detection of molecules of interest, especially for the detection of analytes in liquid medium.

According to another embodiment form, another subject of the invention is the use of at least one trifunctional reagent as described previously, for the preparation of a probe intended for functional proteomics.

Another subject of the present invention is therefore a probe for functional proteomics, characterized by the fact that it is constituted of a trifunctional reagent comprising:
a group that enables the detection (or visualization) and/or the purification of a target protein (reporter tag),
a unit recognized by said target protein (recognition unit), and
a reactive group that enables a covalent bond to be established with the active site of the target protein (reactive group).

According to the invention, the expression “unit recognized by said target protein” is understood to mean any ligand of the target protein or any substrate when it is an enzyme (peptide sequence for example).

According to one preferred embodiment form of the invention, the target protein is an enzyme.

According to a first embodiment form of these probes for functional proteomics and when the target protein is an enzyme, the unit recognized by the enzyme and the reactive group permitting a covalent bond with the active site of this enzyme belong to the same entity (this is the case, for example, for irreversible inhibitors and/or suicide substrates used in medicinal chemistry) and are therefore borne by the same unit of the pseudopeptide trifunctional reagent in accordance with the invention. In this case, the other two units of said trifunctional reagent may be used to attach a group that enables detection (fluorophore for example) and a group that facilitates purification (biotin, polyhistidine-tag, etc.). According to this embodiment form, the three entities may be attached indifferently to any unit of the pseudopeptide trifunctional reagent in accordance with the invention.

According to a second embodiment form of these probes for functional proteomics and when the target protein is an enzyme, the unit recognized by the enzyme and the reactive group do not belong to the same entity and are therefore borne by two different units of the pseudopeptide trifunctional reagent in accordance with the invention. In this case, it is preferable to attach them to the two units closest to one another (B and C) so that the reaction of the reactive group definitely takes place in the targeted (active) site. Thus, the last unit of the pseudopeptide trifunctional reagent in accordance with the invention will be used for attaching the group that enables detection and/or purification.

Besides the preceding provisions, the invention also comprises other provisions which will emerge from the description that follows, which refers to examples of the synthesis of trifunctional reagents in accordance with the invention.

### EXAMPLE 1

### Preparation of a Trifunctional Reagent in Accordance with the Invention

Described in this example is the synthesis of a trifunctional reagent (I-1) in accordance with the present invention and that corresponds to the formula (I-1) below:

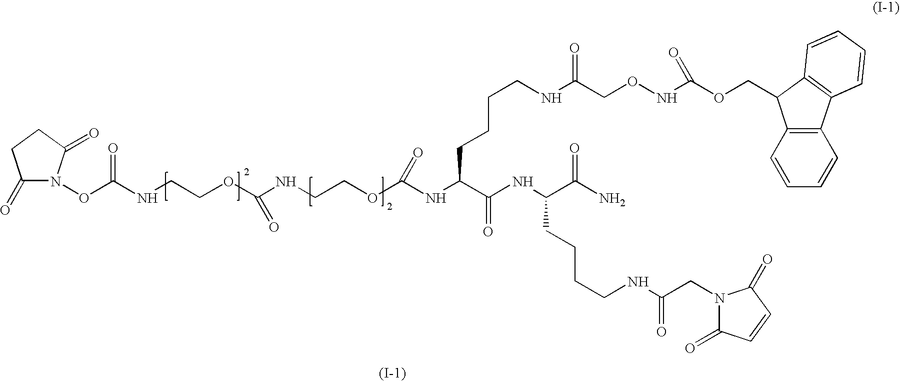

in which:
the unit A is a linker of pseudo-PEG type possessing an activated carbamate unit that is reactive with respect to compounds possessing a primary amine functional group,
the unit B is a lysine bearing on its side chain an oxyamine functional group, protected by a Fmoc group, which oxyamine functional group is reactive with respect to surfaces possessing aldehyde functional groups, and
the unit C is a lysine possessing on its side chain a maleimide unit, which is reactive with respect to compounds possessing a thiol functional group.

The strategy for the synthesis of the compound (I-1) consists in separately preparing three correctly protected precursors (A-1), (B-1) and (C-1), then in assembling them via coupling reactions in order to obtain the trifunctional reagent of formula (I-1) above in accordance with the present invention.

### 1) Synthesis of the Precursor (A-1): Linker Arm of Boc-PEG Type

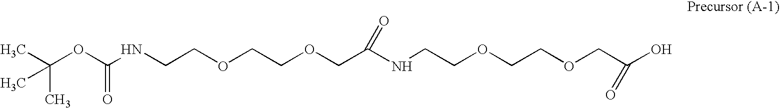

The precursor (A-1) is a hydrophilic linker possessing four ethylene glycol units and also a carboxylic acid functional group which will enable the subsequent coupling with the (B-1) and (C-1) units and a primary amine functional group that it is essential to keep protected until the last step of conversion to “activated carbamate”. It results from the combination of two molecules of 8-amino-3,6-dioxaoctanoic acid, said molecules having been prepared according to the methods described by Rensen, P. C. N. et al., J. Med. Chem., 2004, 47, 5798-5808; Dondoni, A. et al., J. Org. Chem., 2005, 70, 5508-5518 or Dhawan, R. et al. Bioconjugate Chem., 2000, 11, 14-21. This amino acid is then converted into two protected derivatives (N-Boc (6) and methyl ester (7) derivative) according to the methods described, for example, by Nakatani, K. et al., Bioorg. Med. Chem. Lett., 2004, 14, 1105-1108 or Rachele, J., J. Org. Chem., 1963, 28, 2898, which were able to be coupled together according to the method described, for example, by Han, S.-Y. et al., Tetrahedron, 2004, 60, 2447-2467 in order to result, after saponification, in the desired precursor (A-1).

### a) First Step: Synthesis of 2-(2-(2-azido-ethoxy)ethoxy)ethanol (3)

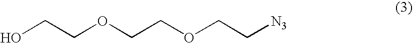

1.29 ml (8.9 mmol) of 2-(2-(2-chloro-ethoxy)ethoxy)ethanol were added to a suspension of sodium azide (0.7 g, 10.7 mmol) and of sodium iodide (0.14 g, 0.93 mmol) in anhydrous ethanol. The resulting yellow mixture was heated under reflux for 5 days under an argon atmosphere. Thin layer chromatography (TLC), supplies sold under the reference DC Kieselgel 60 F254 by Merck, using a dichloromethane/methanol (9:1, v/v) solvent mixture was used to verify that the reaction was complete. The mixture was then filtered over Celite® 545 in order to remove the sodium salts, then evaporated. The resulting oily residue was dissolved in around 10 ml of dichloromethane, then stored at a temperature of 4° C. for 1 hour. After filtration through cotton wool and concentration, the expected compound (3) was obtained in the form of a colorless oil (quantitative yield).

¹H NMR analysis (300 MHz, CDCl₃): δ=2.63 (t, J=6.0 Hz, 1H, OH), 3.43 (t, J=4.9 Hz, 2H), 3.61-3.77 (m, 10H).

b) Second Step: Synthesis of 2-(2-(2-azido-ethoxy)ethoxy)acetic acid (4)

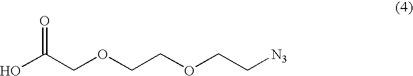

1.56 g (8.9 mmol) of the compound (3) obtained above in the preceding step were dissolved in 90 ml of acetone and the resulting solution was cooled to a temperature of 4° C. 8.9 ml of 3 M Jones reagent that was freshly prepared (for example by dissolving 26.72 g of CrO₃ in 23 ml of concentrated sulfuric acid, then adding water up to a volume of 100 ml) were then added dropwise (a green precipitate was immediately formed), then the resulting reaction mixture was stirred at ambient temperature for 1 hour. TLC was used to check that the reaction was complete as in the preceding step and the reaction was stopped by addition of around 4 ml of propan-2-ol. After 15 min, 100 ml of acetone were added and the green precipitate of Cr(III) salts was removed by filtration over Celite® 545. The filtrate was then evaporated to dryness. The resulting oily residue was purified by chromatography on a silica gel (50 g) column using, as the mobile phase, a gradient of methanol (0 to 5%) in dichloromethane. 1.53 g (8.1 mmol) of the compound (4) were obtained in the form of a yellow oil (91% yield).

¹H NMR analysis (300 MHz, CDCl₃): δ=3.43 (t, J=4.9 Hz, 2H), 3.66-3.80 (m, 6H), 4.19 (s, 2H).

¹³C NMR analysis (75.5 MHz, CDCl₃): δ=50.7; 68.6; 70.2; 70.6; 71.4; 174.2.

### c) Third Step: Synthesis of 2-(2-amino-ethoxy)ethoxy)acetic acid (5)

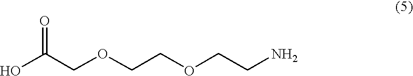

A mixture of 1.53 g (8.2 mmol) of the compound (4) obtained above in the preceding step and of 2.5 ml (66.5 mmol) of formic acid was dissolved in 150 ml of ethanol, then the solution obtained was cooled to a temperature of 4° C. 0.32 g of palladium/charcoal (Pd/C) containing 10% of Pd were added to this solution and the resulting reaction mixture was stirred at ambient temperature for 12 hours under a hydrogen atmosphere. It was then verified that the reaction was complete by TLC as in the preceding step but using an 8:2 (v/v) mixture of dichloromethane (CH₂Cl₂) /methanol (MeOH), then the mixture was filtered over Celite® 545 in order to remove the Pd/C. The filtrate was then evaporated to dryness until an oily residue was obtained which was dried under vacuum in order to result in the expected compound (5) in the form of a yellow oil (quantitative yield).

### d) Fourth Step: Synthesis of the Methyl Ester of 2-(2-aminoethoxy)ethoxy)acetic acid (6)

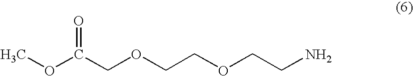

0.48 g (2.92 mmol) of the compound (5) obtained above in the preceding step were put into suspension in 20 ml of 2,2-dimethoxypropane, then 2.92 ml of concentrated hydrochloric acid (37% HCl) were added. The resulting reaction mixture was kept stirring at ambient temperature for 1 hour. It was then verified that the reaction was complete by TLC using an 80:20:2 (v/v/v) CH₂Cl₂/MeOH/triethylamine mixture, then the mixture was evaporated to dryness. The resulting oily residue then underwent four successive lyophilization operations with addition of 10 ml of osmosed water each time in order to finally obtain the expected product in the form of a yellow oil (quantitative yield).

¹H NMR analysis (300 MHz, CD₃CN+5% D₂O): δ=3.08 (t, J=5.3 Hz, 2H), 3.56-3.76 (m, 11H), 4.13 (s, 2H).

### e) Fifth Step: Synthesis of 2-(2-tert-butyl-oxycarbonyl)aminoethoxy)ethoxy)acetic acid (7)

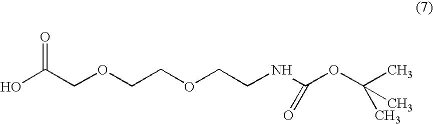

0.78 g (3.75 mmol) of the compound (5) obtained above in step c) were dissolved in 12 ml of a tetrahydrofuran (THF)/H₂O (2:1, v/v) mixture. 5 ml of a freshly prepared 2 M aqueous solution of sodium hydroxide were then added and the solution obtained was cooled to 4° C. 0.89 g (4.12 mmol) of di-t-butyl dicarbonate were then added and the reaction mixture was kept stirring at ambient temperature for 1 hour. It was then verified that the reaction was complete by TLC using a CH₂Cl₂/MeOH (7:3, v/v) mixture. The reaction mixture was then acidified by addition of around 25 ml of a 1 M aqueous solution of potassium hydrogen sulfate (KHSO₄). This solution was then extracted with three lots of 50 ml of ethyl acetate. The organic phase was dried over sodium sulfate (Na₂SO₄) then evaporated to dryness. The resulting oily residue was purified by chromatography on a silica gel (40 g) column using, as the mobile phase, a gradient of methanol (0 to 6%) in CH₂Cl₂. 0.50 g (1.87 mmol) of the compound (7) was obtained in the form of a colorless oil (50% yield).

¹H NMR analysis (300 MHz, CDCl₃): δ=1.44 (s, 9H), 3.34 (bm, 2H), 3.50-3.77 (m, 6H), 4.17 (s, 2H), 4.97 (bs, 1H, NH).

### f) Fifth Step: Synthesis of the Precursor (A-1)

0.15 g (0.83 mmol) of the compound (7) obtained above in the preceding step and 0.23 g (0.87 mmol) of the compound (6) obtained above in step d) were dissolved in 8 ml of anhydrous acetonitrile. Next, 0.44 ml (2.5 mmol) of N,N-diisopropylethylamine (DIEA) and 0.37 g (0.83 mmol) of benzotriazol-1-yl-N-oxytris(dimethylamino)phosphonium hexafluorophosphate (BOP) were added then the reaction mixture was kept stirring at ambient temperature overnight under an argon atmosphere. It was then verified that the reaction was complete by TLC using a CH₂Cl₂/MeOH (8:2, v/v) mixture, then the reaction mixture was evaporated to dryness. The resulting residue was then taken up in 75 ml of ethyl acetate, washed successively with 50 ml of a 10% citric acid aqueous solution, 50 ml of a saturated solution of sodium bicarbonate (NaHCO₃) and 50 ml of a saturated aqueous solution of NaCl in order to then be dried over Na₂SO₄ and evaporated to dryness. The orange oily residue was then dissolved in 5 ml of MeOH and the solution was cooled to 4° C. 0.83 ml of a 1 M aqueous solution of sodium hydroxide were then added and the reaction mixture was kept stirring at ambient temperature for 30 min. It was then verified that the reaction was complete by TLC using a CH₂Cl₂/MeOH (7:3, v/v) mixture. The reaction mixture was then acidified by addition of around 1 ml of a 1 M aqueous solution of KHSO₄. The solution was then evaporated to dryness without heating and the resulting residue was purified by chromatography on a silica gel (30 g) column using, as the mobile phase, a gradient of methanol (0-50%) in CH₂Cl₂. Thus, 0.17 g (0.42 mmol) of the precursor of the linker (A-1) was obtained in the form of a colorless oil with a yield of 80%.

¹H NMR analysis (300 MHz, CDCl₃); δ=1.43 (s, 9H), 3.30 (bm, 2H), 3.47-3.66 (m, 14H), 4.01 (s, 2H), 4.04 (s, 2H), 5.16 (bs, 1H, NH), 7.34 (bs, 1H, NH).

¹³C NMR (75.5 MHz, CDCl₃); δ=28.8 (3C); 38.9; 70.3 (3C); 70.5 (3C); 71.1 (3C); 79.8; 156.5; 171.6; 175.0.

MS (ESI, positive mode) m/z: 431.27 (M+Na)⁺, 453.27 (M−H+2Na)⁺.

MS (ESI, negative mode) m/z: 407.47 (M−H) calculated for C₁₇H₃₂N₂O₉ 408.45

### 2) Synthesis of the Precursor (B-1)

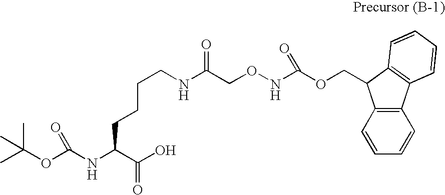

The precursor (B-1) was synthesized in two steps starting from the N-Fmoc protected derivative of aminooxyacetic acid (8) previously prepared by reaction between commercial aminooxyacetic acid and 9-fluorenylmethanol chloroformate under the Schotten-Baumann conditions (Cipolla L. et al., Bioorg. Med. chem., 2002, 10, 1639-1646).

### a) First Pre-Step: Synthesis of 9-fluorenylmethoxy-carbonylaminooxyacetic acid (8)

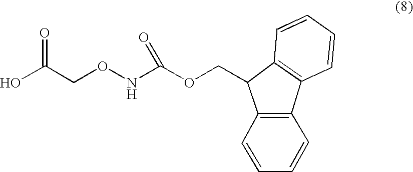

0.5 g (4.6 mmol) of carboxymethoxyamine hemichlorohydride were dissolved in 20 ml of an aqueous solution containing 1.2 g of Na₂CO₃, then the resulting solution was cooled to 4° C. 1.31 g (5.0 mmol) of 9-fluorenylmethyl chloroformate in 10 ml of anhydrous dioxane were then added dropwise to the reaction medium, which then was kept stirring at ambient temperature overnight. The reaction mixture was concentrated, acidified to pH 4-5 by addition of around 5 ml of a 5% aqueous hydrochloric acid solution and the product was precipitated in the solution. It was recovered by filtration and washed once with 20 ml of distilled water then once with 20 ml of pentane. The residual water was removed by lyophilization in order to result in 1.05 g (3.4 mmol) of the compound (8) in crude form. After purification by chromatography on a silica gel (30 g) column using, as the mobile phase, a gradient of methanol (0-50%) in dichloromethane, 0.64 g (2.0 mmol) of the compound (8) was obtained in the form of a white foam (43% yield).

¹H NMR analysis (300 MHz, CD₃OD): δ=4.22-4.27 (m, 3H), 4.44 (d, J=6.8 Hz, 2H), 7.29-7.42 (m, 4H), 7.64 (d, J=7.5 Hz, 2H), (d, J=7.5 Hz, 2H).

### b) Second Pre-Step: Synthesis of N^{α}-(tert-butyloxy-carbonyl)-L-lysine (10)

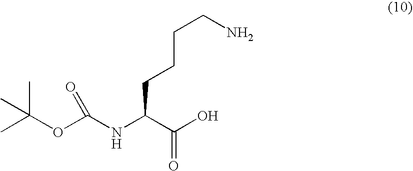

1.0 g (2.63 mmol) of e-(tert-butyloxycarbonyl)-N^{ε}-(benzyloxycarbonyl))-L-lysine (commercial product) was dissolved in 50 ml of ethyl acetate then the solution was cooled to 4° C. Next, 0.1 g of Pd/C containing 10% Pd was added, then the resulting reaction mixture was kept stirring at ambient temperature for 21 hours under a hydrogen atmosphere. It was verified that the reaction was complete by TLC (CH₂Cl₂/MeOH 85:15, v/v) then the reaction mixture was filtered over Celite® 545 in order to remove the Pd/C. Due to the low solubility of the compound (10) in ethyl acetate, the Celite® 545 was washed with 2 lots of 50 ml of methanol in order to recover the compound (10). The filtrate was evaporated to dryness and the oily residue obtained was dried under vacuum in order to result in 0.41 g (1.67 mmol) of the compound (10) in the form of a white foam with a yield of 64%.

### c) First Step: Synthesis of the Succinimidyl Ester of 9-fluorenylmethoxycarbonylaminooxyacetic acid (9)

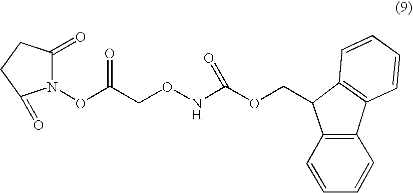

0.560 g (1.80 mmol) of the compound (8) obtained above in step a) were dissolved in 15 ml of an ethyl acetate/dioxane (2:1, v/v) mixture, then the solution was cooled to a temperature of 4° C. 0.225 g (1.95 mmol) of N-hydroxysuccinimide and 0.404 g of N,N′-dicyclohexylcarbodiimide (DCC) were added successively to this solution and the resulting reaction medium was kept stirring at ambient temperature overnight under an argon atmosphere. The conversion of the compound (8) to the corresponding N-hydroxysuccinimidyl ester (9) was verified by reversed phase high performance liquid chromatography (RP-HPLC) on a Thermo Hypersil GOLD®C₁₈ column of 5 μm and of dimensions 4.6×150 mm, using acetonitrile and a 0.1% (v/v, pH 2) aqueous solution of trifluoroacetic acid (TFA) as eluents (80% of 0.1% aqueous solution of TFA with a linear gradient from 20 to 90% of acetonitrile every 35 minutes, at a flow rate of 1.0 ml/min. A double detection UV analysis was carried out at 210 and 254 nm. The compound (9) (t_{R}=20.72 min) was then immediately used in the following coupling reaction without purification.

### d) Second Step: Synthesis of N^{α}-(tert-butyl-oxycarbonyl)-N^{α}-(9-fluorenylmethoxycarbonylamino-oxyacetyl)-L-lysine (B-1)

Added successively to a suspension of N^{α}-Boc-L-lysine (10) (0.41 g, 1.67 mmol) in 6 ml of an anhydrous DMF/NMP (5:1, v/v) mixture were DIEA (0.29 ml, 1.67 mmol) and the crude N-hydroxysuccinimide ester solution (9) obtained above in the preceding step. The resulting reaction medium was stirred at ambient temperature for 30 minutes. An aqueous solution of DIEA (0.15 ml in 5 ml) was then added and the resulting solution was stirred at ambient temperature for 90 minutes. The reaction was monitored by RP-HPLC (system identical to that used above in the preceding step for the analysis of the compound (9)). The reaction mixture was acidified to pH 5-6 by addition of a 5% aqueous solution of HCl (˜3 ml) then evaporated to dryness. The residue thus obtained was taken up in 50 ml of ethyl acetate and the insoluble solid residue (precipitate of DCU) was removed by a filtration over Celite® 545. The filtrate was washed with 30 ml of a 10% aqueous solution of citric acid, dried over Na₂SO₄, then evaporated to dryness. The oily yellow residue thus obtained was purified by chromatography on a silica gel (45 g) column using, as the mobile phase, a gradient of MeOH (0-6%) in dichloromethane. 310 mg (0.57 mmol) of compound (B-1) were obtained in the form of a white foam with a yield of 34%.

¹H NMR analysis (300 MHz, CD₃CN): δ=1.37-1.83 (m, 15H), 3.18 (q, J=6.4 Hz, J=12.6 Hz, 2H), 4.00 (m, 1H, α-CH), 4.04 (s, 2H), 4.26 (t, J=6.8 Hz, 1H), 4.49 (d, J=6.8 Hz, 2H), 5.61 (bd, J=7.5 Hz, 1H, NH), 7.31-7.44 (m, 4H), 7.51 (bs, 1H, NH), 7.63 (d, J=7.5 Hz, 2H), 7.83 (d, J=7.5 Hz, 2H), 8.9 (bs, 1H, NH).

¹³C NMR analysis (75.5 MHz, CD₃CN): δ=23.5; 28.5 (3C); 29.5; 31.6; 38.9; 47.7; 54.2; 68.0; 76.3; 79.8; 120.9 (2C); 126.0 (2C); 128.1 (2C); 128.7 (2C), 142.1 (2C); 144.6 (2C); 156.7; 158.9; 169.3; 174.5.

MS (Maldi-TOF, positive mode) m/z: 564.5709 (M+Na)⁺, 580.5535 (M+K)⁺, calculated for C₂₈H₃₅N₃O₈ 541.61.

### 3) Synthesis of the Precursor (C-1)

This synthesis is represented in scheme 1 below:

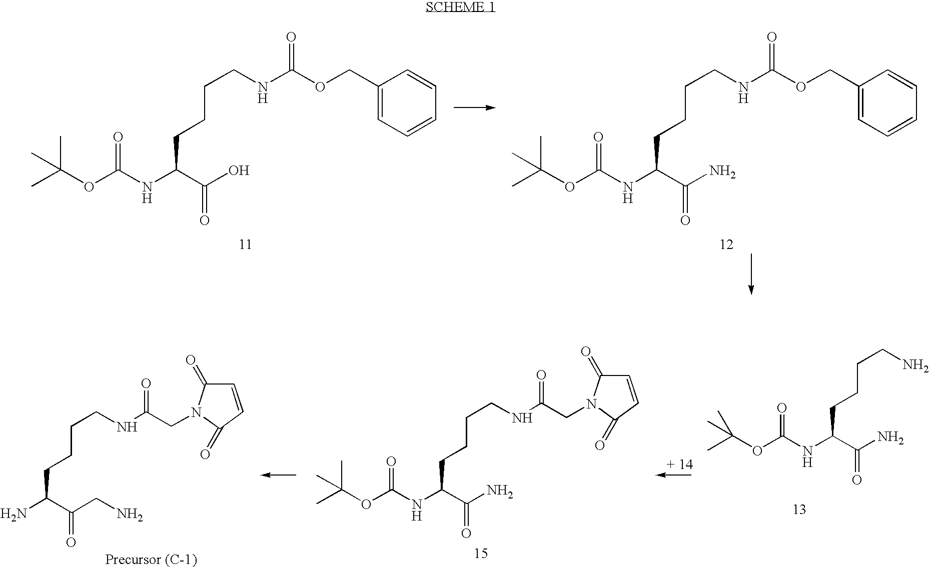

As shown in scheme 1, the precursor (C-1) was synthesized in four steps starting from N^{α}-Boc-N^{α}—Z-L-lysine (11); in this compound Z represents the benzyloxycarbonyl group. Firstly, the carboxylic acid functional group is converted to carboxamide (12) by reaction of ammonia over the mixed anhydride immediately formed according to the method described, for example, by Hofmann, K. et al., J. Am. Chem. Soc., 1978, 100, 3585-3590. Next, the ε-NH₂ functional group protected by a Z group was liberated (13) by catalytic hydrogenation in order to be able to couple the maleimide derivative of glycine (14) previously prepared by reaction between glycine and N-(methyloxycarbonyl)maleimide according to the method described by Keller O. et al., Helv. Chim. Acta, 1975, 58, 531-541. The maleimide derivative (15) was then isolated by chromatography over silica gel with a yield of 51%. Finally, the N-terminal end was deprotected by treatment with trifluoroacetic acid in order to result in the precursor (C-1).

### a) First Step: Synthesis of N^{α}-(tert-butyloxycarbonyl)-N^{ε}-(benzyloxycarbonyl))-L-lysinecarboxyamide (12))

1.0 g (2.63 mmol) of N^{α}-(tert-butyloxycarbonyl-N^{ε}-benzyloxycarbonyl)-L-lysine (11) was dissolved in 18 ml of anhydrous ethyl acetate and the solution was cooled to −15° C. in a bath composed of dry ice (solid CO₂) and of ethylene glycol. 0.29 ml (2.63 mmol) of N-methylmorpholine (NMM) and 0.34 ml (2.63 mmol) of isobutyl chloroformate were then added and the resulting reaction medium was kept stirring at −15° C. for 10 minutes under an argon atmosphere. Next, 1.3 ml of aqueous ammonia (50%; v/v) was added to this mixture and the reaction mixture was kept stirring at a temperature of 0° C. for 1 hour. A bulky white precipitate was immediately formed; this was recovered by filtration and washed with 25 ml of distilled water then with 25 ml of pentane. The residual water was removed by lyophilization in order to result in 0.705 g (1.86 mmol) of the compound (12). The filtrate was furthermore taken up in 20 ml of ethyl acetate, washed with 20 ml of distilled water, dried over Na₂SO₄ and evaporated to dryness. The white solid obtained was washed with 2 lots of 25 ml of pentane and dried under vacuum. A supplementary amount of compound (12) (0.25 g, 0.66 mmol) was thus obtained. The total yield was 95%.

¹H NMR analysis (300 MHz, CD₃CN): δ=1.31-7.78 (m, 15H), 3.10 (q, J=6.4 Hz, J=12.9 Hz, 1H), 3.92 (m, 1H, α-CH), 5.06 (s, 2H, CH₂—Bn), 5.53 (bs, 1H, NH), 5.66 (bs, 1H, NH), 5.71 (bs, 1H, NH), 6.31 (bs, 1H, NH), 7.30-7.42 (m, 5H).

### b) Second Step: Synthesis of N^{α}-(tert-butyl-oxycarbonyl)-L-lysinecarboxyamide (13)

0.95 g (2.5 mmol) of the compound (12) obtained above in the preceding step were dissolved in 50 ml of an ethyl acetate/ethanol (4:1, v/v) mixture and the solution was cooled to 4° C. 0.1 g of Pd/C containing 10% of Pd was added and the resulting reaction mixture was kept stirring at ambient temperature for 3 hours under an argon atmosphere. It was verified that the reaction was complete by TLC (CH₂Cl₂/MeOH 9:1, v/v), then the mixture was filtered over Celite® 545 to remove the Pd/C. The filtrate was then evaporated to dryness in order to result in an oily residue which was then evaporated under vacuum in order to result in the expected compound (13) in the form of a white solid (quantitative yield).

¹H NMR (300 MHz, CD₃₀D): δ=1.40-1.80 (m, 15H), 2.70 (t, J=6.8 Hz, 1H), 4.03 (q, J=4.9 Hz, J=12.1 Hz, 1H, α-CH).

### c) Additional Step: Synthesis of N-maleoyl-L-glycine (14)

### i) Preliminary Step: Preparation of N-(methyloxy-carbonyl) maleimide

1 g (10.3 mmol) of maleimide was dissolved in 20 ml of anhydrous ethyl acetate then the solution was cooled to 4° C. 1.13 ml of NMM (10.3 mmol) and 0.80 ml (10.3 mmol) of methyl chloroformate were then added, then the resulting reaction mixture was kept stirring at ambient temperature for 1 hour. A white-colored bulky precipitate of NMM.HCl was immediately formed; it was removed by filtration and washed with around 20 ml of ethyl acetate. The filtrate was washed with 30 ml of a saturated aqueous solution of NaCl, dried over Na₂SO₄ and evaporated to dryness. The resulting oily residue was dried under vacuum in order to result in N-(methyloxycarbonyl)maleimide in the form of a purple-brown solid (1.16 g, 7.5 mmol, 73% yield).

### ii) Preparation of N-maleoyl-L-glycine (14)

0.5 g of glycine (6.7 mmol) was dissolved in an aqueous solution of NaHCO₃ (2.8 g in 32 ml) and the solution was then cooled to 0° C. (NaCl/ice bath). 1.04 g (6.7 mmol) of N-(methyloxycarbonyl)maleimide obtained in step i) above were then added and the resulting reaction mixture was mixed vigorously at a temperature of 0° C. for 20 minutes. Next, 60 ml of distilled water were added and the resulting aqueous solution was left at ambient temperature overnight. The reaction mixture was then acidified to pH 6 by addition of H₂SO₄ then partially evaporated. A supplementary amount of H₂SO₄ was then added in order to achieve a pH of 1-2, then the aqueous solution was extracted using ethyl acetate (2×50 ml). The organic phase was washed with a saturated aqueous solution of NaCl (50 ml), dried over Na₂SO₄ and evaporated to dryness. The resulting oily residue was purified by chromatography over a silica gel (25 g) column using, as the mobile phase, a mixture of CHCl₃/AcOH (95:5, v/v). Finally, the traces of AcOH were removed by lyophilization. The N-maleoyl-L-glycine (14) was then obtained in the form of a colorless solid (0.61 g, 3.6 mmol, 54% yield). This compound (14) was then immediately used in the third step which is described below.

¹H NMR analysis (300 MHz, acetone-d₆): δ=4.29 (s, 2H, CH₂-Gly), 7.02 (s, 2H, CH-Mal).

¹³C NMR analysis (75.5 MHz, acetone-d₆): δ=39.6, 136.3 (2C), 169.8 (2C), 171.7 (1C).

### d) Third Step: Synthesis of N^{α}-(tert-butyloxycarbonyl)-N^{ε}-(N-maleoyl-L-glycyl)-L-lysinecarboxyamide (15)

0.29 g (1.73 mmol) of N-maleoyl-L-glycine (14) obtained previously was dissolved in 5 ml of anhydrous CH₃CN. A solution of N^{α}-Boc-L-lysine-NH₂ (11) (0.55 g, 2.24 mmol) in 5 ml of anhydrous DMF was added and the resulting solution was cooled to 4° C. 0.23 g (1.73 mmol) of hydroxybenzotriazole monohydrate (HOBt) and 0.39 g (1.90 mmol) of DCC were added. The resulting reaction mixture was kept stirring at ambient temperature overnight under an argon atmosphere. It was verified that the reaction was complete by TLC (CH₂Cl₂/MeOH 8:2, v/v) and the mixture was filtered over Celite® 545 in order to remove the white precipitate of N,N′-dicyclohexylurea (DCU). The filtrate was then evaporated to dryness, then dried under vacuum to remove the DMF. The resulting residue was then taken up in 50 ml of ethyl acetate, washed with 30 ml of saturated NaHCO₃, 30 ml of a saturated aqueous solution of NaCl, then dried over Na₂SO₄ and evaporated to dryness. The resulting residue was purified by chromatography over a silica gel (30 g) column using, as the mobile phase, a gradient of methanol (0-15%) in dichloromethane, in order to result in 0.35 g (0.88 mmol) of the expected compound (15) in the form of a white solid with a yield of 51%.

IR analysis (KBr) Vₘₐₓ 835, 1063, 1166, 1256, 1432, 1525, 1555, 1665 (broad), 1710 (broad), 2926, 3218, 3342 (broad), 3416 cm⁻¹.

¹H NMR analysis (300 MHz, CD₃CN): δ=1.29-1.78 (m, 15H), 3.15 (q, J=6.4 Hz, J=13.0 Hz, 1H), 3.88-3.95 (m, 1H, α-CH), 4.05 (s, 2H), 5.52 (bs, 1H, NH), 5.72 (bs, 1H, NH), 6.34 (bs, 1H, NH), 6.62 (bs, 1H, NH), 6.85 (s, 2H).

¹³C NMR analysis (75.5 MHz, CD₃CN): δ=24.1, 29.0 (3C), 30.1, 33.0, 40.1, 41.4, 55.7, 80.2, 136.1 (2C), 157.1, 167.8, 172.1, 176.0.

### e) Fourth Step: Synthesis of N^{ε}-(N-maleoyl-L-glycyl)-L-lysinecarboxamide (precursor C-1)

0.33 g (0.83 mmol) of N^{α}-(tert-butyloxycarbonyl)-N^{ε}-(N-maleoyl-L-glycyl)-L-lysinecarboxamide (15) was dissolved in 7 ml of a TFA/H₂O mixture (95:5, v/v) then the solution was left at ambient temperature for 90 minutes. The TFA was then evaporated and the product was precipitated with ether, washed with ether and lyophilized. The crude TFA salt of the precursor (C-1) was purified by reversed-phase flash chromatography over a C₁₈ grafted silica column (20 g, elution with a 0.1% aqueous solution of TFA). The fractions containing the product were lyophilized in order to give 91 mg (0.22 mmol, 27% yield) of the expected precursor (C-1) in the form of a white solid.

¹H NMR analysis (300 MHz, D₂O): δ=1.33-1.42 (m, 2H), 1.49-1.59 (m, 2H), 1.83-1.90 (m, 2H), 3.21 (t, J=6.8 Hz, 2H), 3.98 (t, J=6.8 Hz, 1H, α-CH), 4.22 (s, 2H), 6.92 (s, 2H).

4) Synthesis of the Non-Fluorescent Functional Reagent (I-1) in Accordance with the Invention:

### Assembly of the Precursors (A-1), (B-1) and (C-1)

The trifunctional compound (I-1) was synthesized in 5 steps from the precursors (A-1), (B-1) and (C-1) prepared above during the preceding steps.

The protocol for the synthesis of the compound (I-1) is summarized (the three main steps only) in scheme 2 below:

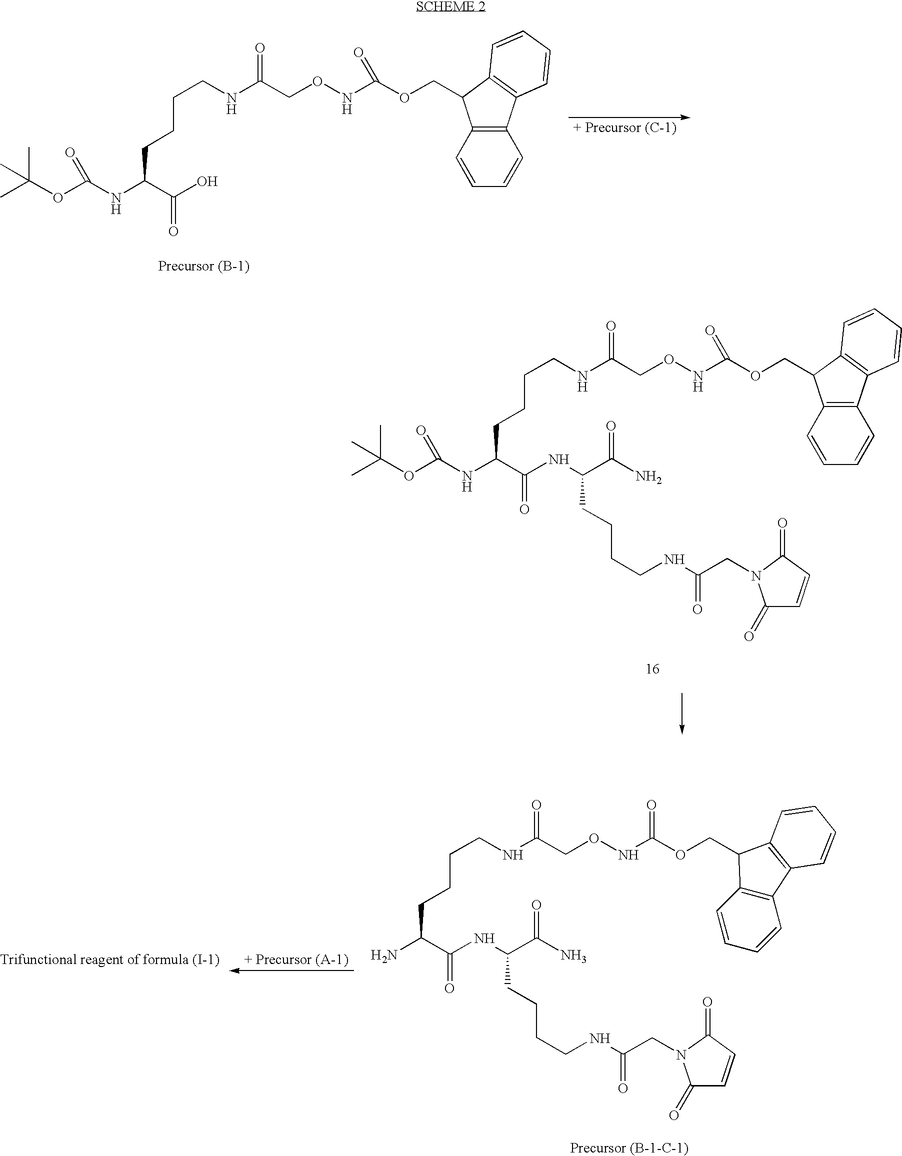

The coupling between the precursors (B-1) and (C-1) was carried out after preactivation of the precursor (B-1) in the form of hydroxysuccinimide ester according to the method described for example in the article by Knorr, R. et al., Tetrahedron Lett., 1989, 30, 1927-1930. The activated ester was then reacted with the precursor (C-1) in order to result in a coupling product (16) which was isolated by chromatography over silica gel with a yield of 32%. Next, treatment with TFA made it possible to remove the Boc group and to thus obtain the precursor (B-1-C-1). The final coupling between the precursor (A-1) and the precursor (B-1-C-1) was carried out using BOP as a coupling reagent. The expected product, in the still protected form, was purified by chromatography over silica gel. After deprotection, by treatment with trifluoroacetic acid, the N-terminal end was converted to activated carbamate by treatment with N,N′-disuccinimidyl carbonate (DSC) in anhydrous DMF in the presence of triethylamine. A total conversion of the pseudopeptide to a compound of formula (I-1) was observed at the end of 30 minutes. The expected compound of formula (I-1) was then purified by reversed-phase flash chromatography over a C₁₈ grafted silica column.

### a) First Step: Synthesis of N^{α}-(tert-butyloxycarbonyl)-N^{ε}-(9-fluorenylmethyloxycarbonylaminooxyacetyl)-L-lysine-N^{ε}-maleoyl-L-lysinecarboxamide (16)

0.2 g (0.36 mmol) of the precursor (B-1) obtained above in step 2) d) was dissolved in 2 ml of anhydrous acetonitrile. Next, 67.4 μl (0.38 mmol) of DIEA and 116 mg (0.38 mmol) of O—(N-succinimidyl)-N,N,N′,N′-tetramethyluronium tetrafluoroborate (TSTU) were added and the reaction mixture was kept stirring at ambient temperature for 45 minutes under an argon atmosphere. The conversion of the precursor (B-1) to its N-hydroxysuccinimide ester was verified by TLC (CH₂Cl₂/MeOH, 85:15, v/v). A solution of 91 mg (0.23 mmol) of N^{α}—(N-maleoyl-L-glycyl)-L-lysine-carboxamide (precursor C-1) obtained above in step 3 e) and of 40 μl (0.23 mmol) of DIEA in 3 ml of an anhydrous CH₃CN/DMF mixture (2:1, v/v) were then added dropwise and the resulting reaction mixture was kept stirring at ambient temperature for 1 hour under an argon atmosphere. It was verified that the reaction was complete by a TLC (CH₂Cl₂/MeOH, 85:15, v/v) then the mixture was evaporated under vacuum in order to remove the DMF. The oily residue thus obtained was purified by chromatography over a silica gel (25 g) column using, as the mobile phase, a gradient of MeOH (0-10%) in dichloromethane. Thus, 60 mg (0.075 mmol) of the compound (16) were obtained in the form of a white foam with a yield of 32%.

¹H NMR analysis (300 MHz, CDCl₃): δ=1.25-1.91 (m, 21H), 3.07-3.29 (m, 4H), 4.08-4.42 (m, 7H, 2×α-CH, CH-Fmoc, 2×CH₂), 4.48 (d, J=6.8 Hz, 2H, CH₂-Fmoc), 5.61 (bd, J=6.4 Hz, 1H, NH), 6.04 (bs, 1H, NH), 6.74 (s, 2H), 6.81 (bs, 1H, NH), 6.91 (bs, 1H, NH), 7.27-7.42 (m, 4H), 7.57 (d, J=7.5 Hz, 2H), 7.75 (d, J=7.5 Hz, 2H), 9.2 (bs, 1H, NH).

MS (MALDI-TOF, positive mode) m/z 706.6896 ((M-Boc)+H)⁺ 808.7330 (M+H)⁺, 828.7064 (M+Na)⁺, calculated for C₄₀H₅₁N₇O₁₁ 805.89.

### b) Second Step: Synthesis of N^{ε}-(9-fluorenyl-methyloxycarbonylaminooxyacetyl)-L-lysine-N^{ε}-maleoyl-L-glycyl)-L-lysinecarboxamide (B-1-C-1)

60 mg (0.075 mmol) of the compound (16) obtained above in the preceding step were dissolved in 2 ml of dichloromethane and the solution was cooled to 4° C. 306 μl (4.12 mmol) of TFA were added dropwise and the resulting reaction mixture was kept stirring at ambient temperature for 90 minutes. It was verified that the reaction was complete by TLC (CH₂Cl₂/MeOH, 85:15, v/v) then the mixture was evaporated to dryness. The oily residue thus obtained was dissolved in 5 ml of a CH₃CN/H₂O mixture (1:1, v/v) then lyophilized in order to give 58 mg (0.071 mmol) of the compound (B-1-C-1) in the form of a white powder (94% yield).

MS (MALDI-TOF, positive mode) m/z 706.6488 (M+H)⁺, calculated for C₃₅H₄₃N₇O₉ 707.77.

This compound was then used in the following step without purification.

### c) Third Step: Synthesis of the Trifunctional Reagent (I-1) in Completely Protected Form

58 mg (0.071 mmol) of the compound (B-1-C-1) obtained above in the preceding step and 32 mg (0.077 mmol) of the precursor (A-1) obtained previously in step 1) f) were dissolved in 2 ml of anhydrous dichloromethane. Next, 25.9 μl (0.15 mmol) of DIEA and 34.4 mg (0.077 mmol) of BOP were added and the reaction mixture was kept stirring at ambient temperature for 30 minutes under an argon atmosphere. It was verified that the reaction was complete by RP-HPLC (System B, that is to say using a Thermo Hypersil GOLD®C₁₈ column of 5 μm and of dimensions 4.6×150 mm with, as eluent, acetonitrile and a 0.1% (v/v, pH 2) aqueous solution of TFA by passing the mixture containing 80% of TFA for 5 minutes, then a linear gradient ranging from 20 to 90% of CH₃CN over 35 minutes with a flow rate of 1.0 ml/min; a double detection UV analysis was carried out at 210 and 254 nm), then a supplementary amount of precursor (A-1) (14.4 mg, 0.035 mmol) of DIEA (18.5 μl, 0.10 mmol) and of BOP (15.6 mg, 0.077 mmol) was added. After mixing at ambient temperature for 90 minutes, the reaction mixture was dissolved in 25 ml of dichloromethane. The resulting organic phase was washed successively with 25 ml of 10% citric acid and 25 ml of brine, then dried over Na₂SO₄ and evaporated to dryness. The oily residue thus obtained was purified by chromatography over a silica gel (10 g) column using, as the mobile phase, a gradient of methanol (0-15%) in dichloromethane in order to result in 24 mg (0.022 mmol) of the completely protected compound of formula (I-1) in the form of a yellow foam with a yield of 31%.

### d) Fourth Step: Synthesis of the TFA Salt of the Completely Protected Compound of Formula (I-1)

24 mg (0.022 mmol) of the compound of formula (I-1) in completely protected form and as obtained above in the preceding step were dissolved in 1 ml of dichloromethane. 81 μl (1.08 mmol) of TFA were then added dropwise, then the resulting reaction mixture was kept stirring at ambient temperature for 1 hour. It was verified that the reaction was complete by TLC (CH₂Cl₂/MeOH, 85:15, v/v) then the mixture was evaporated to dryness. The crude TFA salt of the compound of formula (I-1) in completely protected form was purified by reversed-phase flash chromatography over a C₁₈ grafted silica column (5 g) using, as the mobile phase, a gradient of CH₃CN (0-23%) in a 0.1% aqueous solution of TFA. The fractions containing the product were lyophilized in order to result in 12 mg (0.011 mmol) of the TFA salt of the completely protected trifunctional reagent of formula (I-1) in the form of a white foam.

### e) Fifth Step: Synthesis of the Trifunctional Reagent of Formula (I-1)

12 mg (0.011 mmol) of the TFA salt of the trifunctional reagent of formula (I-1) in completely protected form as obtained above in the preceding step were dissolved in 200 μl of anhydrous DMF. Next, 1.5 μl (0.011 mmol) of TEA and a solution of the reagent DSC (4.15 mg, 0.016 mmol) in 100 μl of anhydrous DMF were added. The reaction mixture then was kept stirring at ambient temperature for 1 hour. It was verified that the reaction was complete by RP-HPLC using the system B as described above in step 4) b). The mixture was then taken up in 5 ml of a 0.1% aqueous solution of TFA and purified by reversed-phase flash chromatography over a C₁₈ grafted silica column (5 g) using, as the mobile phase, a gradient of CH₃CN (0-50%) in a 0.1% aqueous solution of TFA. The fractions containing the product were lyophilized in order to result in 6.70 mg (0.0059 mmol) of the expected trifunctional reagent of formula (I-1) in the form of a white powder.

MS (MALDI-TOF, positive mode) m/z: 996.6590 ((M-NHS carbamate)+H)⁺, 1159.5921 (M+Na)⁺, 1175.5654 (M+K)⁺, calculated for C₅₂H₆₈N₁₀O₁₉ 1137.18.

### EXAMPLE 2

### Synthesis of a Pseudopeptide Trifunctional Reagent in Accordance with the Invention (I-2)

Described in this example is the synthesis of a pseudopeptide trifunctional reagent (I-2) in accordance with the present invention and corresponding to the following formula:

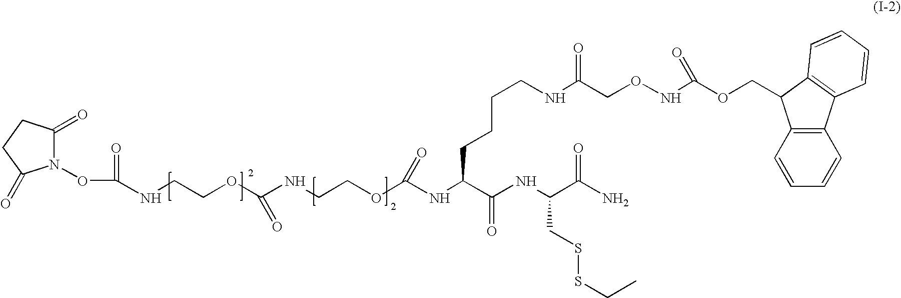

Illustrated in this example is the synthesis of a trifunctional reagent different from the reagent of formula (I-1) previously synthesized above in example 1) by the nature of the amino acid that has to withstand the attachment of a fluorophore (precursor C). This example shows that it is also possible to use a cysteine whose thiol functional group of the side chain has been protected in the form of disulfide using the S-ethyl (SEt) group, with a view to reacting it with a fluorophore previously functionalized by a maleimide or iodoacetyl unit.

In order to do this, a convergent synthesis strategy has been used as in example 1) that consists in separately preparing the precursors (A), (B) and (C) then in assembling them. As precursor (A), the precursor (A-1) synthesized in example 1) above was used. As precursor (B), a precursor of formula (B-1) was synthesized according to a route substantially different and improved relative to that used for the synthesis of the precursor (B-1) from example 1). Finally, the synthesis of the precursor (C-2) was carried out starting from commercial Boc-Cys(SEt)-OH.

### 1) Synthesis of the Precursor (B-1): N^{α}-(tert-butyloxycarbonyl)-N^{ε}-(9-fluorenyloxycarboxylaminooxy-acetyl)-L-lysine

The precursor (B-1) was synthesized from the N-Fmoc protected derivative of aminooxyacetic acid (8) previously prepared as in example 1), step 2) a).

0.230 g (0.73 mmol) of the compound (8) was dissolved in 9 ml of a CH₃CN/DMF mixture (1:1, v/v). Next, 119 mg (0.88 mmol) of hydroxybenzotriazole monohydrate and 182 mg (0.88 mmol) of DCC were added, then the reaction mixture was kept stirring at ambient temperature for 2 hours under an argon atmosphere. A solution of 180 mg (0.731 mol) of N^{α}-Boc-L-lysine (10) as obtained above in step 2), b) of example 1 in 2 ml of anhydrous DMF was then added and the reaction mixture was kept stirring at ambient temperature under an argon atmosphere. The reaction was monitored by TLC (CH₂Cl₂/MeOH, 80:20, v/v). After stirring for 2 hours, the mixture was evaporated to dryness. The residue thus obtained was taken up in 50 ml of ethyl acetate, washed with 50 ml of a 10% aqueous solution of citric acid, 25 ml of distilled water, dried over Na₂SO₄ and finally purified by chromatography on a silica gel (20 g) column using, as the mobile phase, a gradient of ethyl acetate (0-80%) in dichloromethane. 296 mg (0.36 mmol) of compound (B-1) were obtained in the form of a white foam with a yield of 50%.

¹H NMR analysis (300 MHz, CD₃CN): δ=1.37-1.83 (m, 15H), 3.18 (q, J=6.4 Hz, J=12.6 Hz, 2H), 4.00 (m, 1H, α-CH), 4.04 (s, 2H), 4.26 (t, J=6.8 Hz, 1H), 4.49 (d, J=6.8 Hz, 2H), 5.61 (bd, J=7.5 Hz, 1H, NH), 7.31-7.44 (m, 4H), 7.51 (bs, 1H, NH), 7.63 (d, J=7.5 Hz, 2H), 7.83 (d, J=7.5 Hz, 2H), 8.9 (bs, 1H, NH).

¹³C NMR analysis (75.5 MHz, CD₃CH): δ=23.5; 28.5 (3C); 29.5; 31.6; 38.9; 47.7; 54.2; 68.0; 76.3; 79.8; 120.9 (2C); 126.0 (2C); 128.1 (2C); 128.7 (2C), 142.1 (2C); 144.6 (2C); 156.7; 158.9; 169.3; 174.5.

MS (MALDI-TOF, positive mode) m/z: 564.5709 (M+Na)⁺, 580.5535 (M+K)⁺, calculated for C₂₈H₃₅N₃O₈ 541.61.

### 2) Synthesis of the Precursor (C-2)

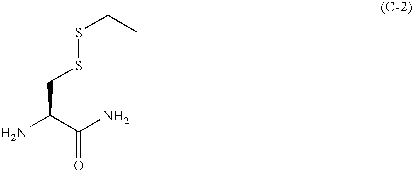

The precursor (C-2) was synthesized in two steps from N-(tert-butyloxycarbonyl)-S—(S-ethyl)cysteine (19) according to the scheme 3 below:

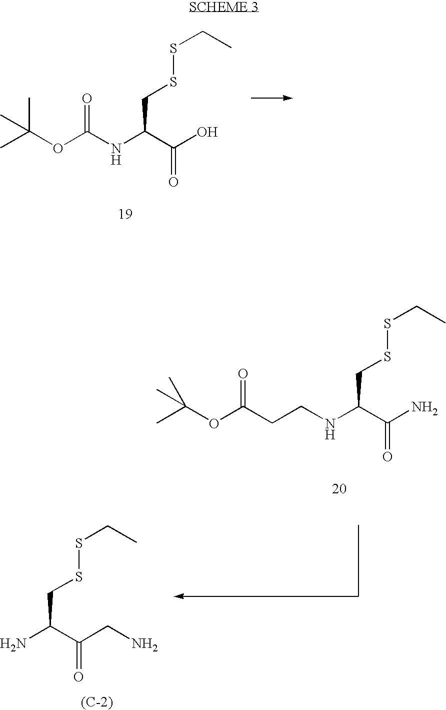

According to this scheme and in a first step, the carboxylic acid functional group of the compound (19) is converted to carboxamide (20) by reaction of aqueous ammonia with the mixed anhydride immediately formed according to the method described for example by Hofmann, K. et al., J. Am. Chem. Soc., 1978, 100, 3585-3590. Next, the N-terminal end of the compound (20) is deprotected by treatment with trifluoroacetic acid in order to result in the precursor (C-2).

### a) First Step: Synthesis of N-(tert-butyloxycarbonyl-S—(S-ethyl)cysteinecarboxamide (6)

500 mg (1.08 mmol) of N-(tert-butyloxycarbonyl)-S—(S-ethyl)cysteine (19) were dissolved in 15 ml of ethyl acetate and the solution was cooled to −15° C. in a bath of dry ice and ethylene glycol. 0.119 ml (1.08 mmol) of NMM and 0.140 mg (1.08 mmol) of isobutyl chloroformate were then added and the reaction mixture was kept stirring at −15° C. for 10 minutes under an argon atmosphere. Added next to this anhydrous solution was an aqueous solution containing 15% ammonia (0.38 ml, 3.24 mmol), then the reaction mixture was kept stirring for 30 minutes at a temperature of 4° C. The mixture was then evaporated to dryness and the residue was taken up in 30 ml of ethyl acetate, then washed with 20 ml of water. The organic phases were dried over Na₂SO₄, then evaporated to dryness. The compound (20) was obtained with a quantitative yield, in the form of a white solid.

¹H NMR analysis (300 MHz, CDCl₃): δ=1.24-1.29 (t, J=6.8 Hz, 3H, CH₃(SEt)), 1.39 (s, 9H, tBu), 2.63-2.70 (q, J=7.1 Hz, 2H, CH₂(SEt)), 2.99-3.01 (d, J=6.0 Hz, 2H, CH₂, β), 4.36-4.39 (m, 1H, CH α), 5.25-5.28 (d, J=9.0 Hz, 1H, NH), 5.63 (bs, 1H, NH), 6.31 (bs, 1H, NH)

¹³C NMR analysis (75.5 MHz, CDCl₃): δ=13.8 (CH₃(SEt)), 27.8 (tBu), 32.0 (CH₂(SEt)), 39.9 (CH₂β), 53.0 (CH α), 80.1 (Cq tBu), 155.1 (Cq), 172.6 (Cq).

MS (MALDI-TOF, positive mode) m/z=303.13 (M+Na)⁺, calculated for C₁₆H₂₀N₂O₃S₂—Na 303.41.

### b) Second Step: Synthesis of the Precursor (C-2): S—(S-ethyl) cysteinecarboxamide

The compound (20) obtained above in the preceding step was slowly dissolved in 14 ml of a TFA/H₂O mixture (95:5, v/v) with stirring at a temperature of between 0° C. and ambient temperature for 1 hour. It was verified that the reaction was complete by TLC (CH₂Cl₂/MeOH 90:10, v/v) then the reaction mixture was evaporated to dryness. A minimal amount of osmosed water was then added and the resulting aqueous solution was lyophilized in order to result in the precursor (C-2) in the form of a yellowish powder with a yield of 89%.

¹H NMR analysis (300 MHz, D₂O): δ=1.26-1.31 (t, J=7.1 Hz, 3H, CH₃(SEt)), 1.39 (s, 9H), 2.72-2.80 (q, J=7.1 Hz, 2H, CH₂(SEt)), 3.25-3.26 (d, J=6.0 Hz, 2H, CH₂ (3), 4.30-4.35 (m, 1H, CH α).

¹³C NMR analysis (75.5 MHz, D₂O): δ=13.8 (CH₃(SEt)), 30.1 (CH₂(SEt)), 38.3 (CH₂β), 52.2 (CH α), 170.8 (Cq).

MS (MALDI-TOF, positive mode) m/z=180.00 (M+Na)⁺, calculated for CH₅H₁₂N₂OS₂.Na 180.29.

### 3) Synthesis of the Trifunctional Reagent of Formula (I-2)

The reagent (I-2) was synthesized in three steps from the precursors (A-1), (B-1) and (C-2) previously synthesized according to scheme 4 below:

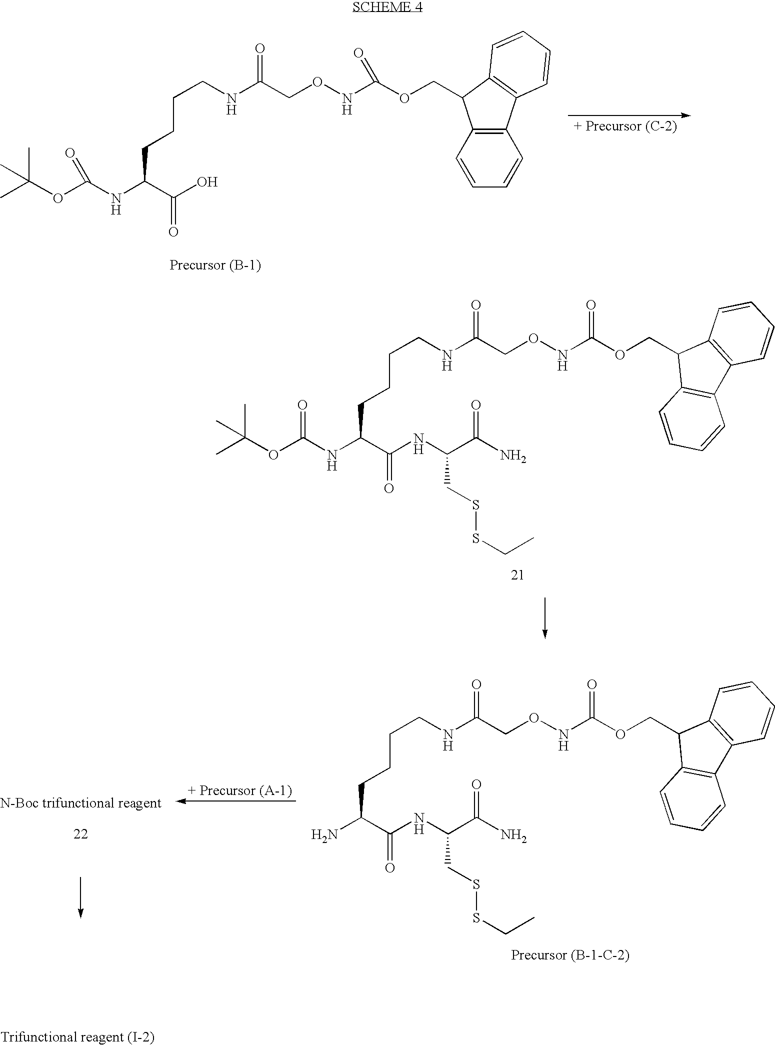

The coupling between the precursors (B-1) and (C-2) was carried out after preactivation of the precursor (B-1) in the form of hydroxybenzotriazole ester according to the method described for example in the article by König, W. et al., Chem. Ber., 1970, 103, 788-798. The activated ester was then reacted with the precursor (C-1) in order to result in a coupling product (21). Since the amines involved were in the TFA salt form, it was necessary to add a base (DIEA). The intermediate compound (B-1-C-2) was purified by chromatography over silica gel with a yield of 61%. The N-terminal end of the N-Boc trifunctional reagent (22) was deprotected with TFA then converted to activated carbamate by treatment using DSC in anhydrous DMF in the presence of TEA.

### a) First Step: Synthesis of N^{α}-(tert-butyloxycarbonyl)-N^{ε}-(9-fluorenylmethyloxycarbonylaminooxyacetyl)-L-lysine-S—(S-ethyl)-L-cysteinecarboxamide (21)

0.196 g (0.36 mmol) of the compound (B-1) obtained above at the end of step 1) was dissolved in 3 ml of an anhydrous CH₃CN/DMF mixture (2:1, v/v). 58.4 mg (0.43 mmol) of hydroxybenzotriazole monohydrate and 89.1 mg (0.43 mmol) of DCC were then added and the resulting reaction mixture was kept stirring at ambient temperature for 2 hours under an argon atmosphere. Next, 1 ml of a solution of DMF containing 106 mg (0.36 mmol) of the compound (C-2) was added and the reaction mixture was kept stirring at ambient temperature under an argon atmosphere. At the end of 2 hours, 31 μl (0.18 mmol) of DIEA were added. At the end of 2 supplementary hours, 31 μl (0.18 mmol) of DIEA were again added. Once again after 2 supplementary hours, 31 μl (0.18 mmol) of DIEA were again added and 44.9 mg (0.18 mmol) of DCC were added. The flask containing the reaction medium was stored at a temperature of −20° C. overnight. The next day, a supplementary amount of DIEA (31 μl, 0.18 mmol) and of DCC (44.9 mg, 0.18 mmol) was added. After 2 hours, the reaction was judged to be complete and the reaction mixture was evaporated to dryness. The resulting residue was taken up in 25 ml of ethyl acetate, washed with 25 ml of a 10% aqueous solution of citric acid, 25 ml of saturated NaHCO₃ and 25 ml of distilled water, dried over Na₂SO₄ and purified by chromatography over a silica gel (20 g) column using, as the mobile phase, a gradient of MeOH (0-10%) in dichloromethane. 222 mg (0.31 mmol) of the compound (21) were obtained in the form of a white foam with a yield of 88%.

¹H NMR analysis (300 MHz, CDCl₃): δ=1.26-1.31 (t, 3H, J=7.1 Hz, CH₃(SEt)Cys), 1.42 (s, 9H, tBu), 1.51-1.92 (m, 6H, CH₂, β, δ, γ Lys), 2.65-2.72 (q, 2H, J=7.5 Hz, CH₂(SEt)Cys), 3.08-3.10 (d, 2H, J=6 Hz, CH₂β Cys), 3.26-3.33 (m, 2H, CH₂ε Lys), 4.04-4.06 (t, J=6.0 Hz, 1H, CH α Lys), 4.20-4.25 (t, J=7.2 Hz, 1H, CH Fmoc), 4.33 (s, 2H), 4.49-4.51 (d, 1H, J=6.4 Hz, CH₂ Fmoc), 4.71-4.78 (q, J=6.4 Hz, 1H, CHα Cys), 7.26-7.78 (m, 8H, CH Fmoc).

¹³C NMR analysis (75.5 MHz, CDCl₃): δ=14.2 (CH₃(SEt) Cys), 22.3 (CH₂γ, Lys) 28.6 (CH₂ δ Lys), 30.1 (CH₂ (SEt) Cys), 32.4 (CH₂β Lys), 38.0 (CH₂β Cys), 39.3 (CH₂ε Lys), 46.7 (CH Fmoc), 52.3 (CH α Cys), 52.2 (CH α Lys), 68.0 (CH₂ Fmoc), 76.1 (CH₂), 80.8 (Cq tBu), 120.3 (Cq Fmoc), 125.1 (Cq Fmoc), 127.4 (Cq Fmoc), 128.1 (Cq Fmoc), 141.4 (Cq), 143.3 (Cq), 156.4 (Cq), 158.6 (Cq), 169.0 (Cq), 172.6 (Cq), 172.8 (Cq).

RP-HPLC (system B): t_{R}=22.3 min, 84% purity

MS analysis (MALDI-TOF, positive mode) m/z=726.77 (M+Na)⁺, 742.74 (M+K)⁺ calculated for C₃₃H₄₅O₈S₂—.Na 726.88.

### b) Second Step: Synthesis of the Coupling Product (B-1-C-2): N^{ε}-(9-fluorenylmethyloxycarbonylaminooxyacetyl)-L-lysine-S—(S-ethyl)-L-cysteinecarboxamide

191 mg (0.27 mmol) of the compound (21) obtained above in the preceding step were dissolved in 7 ml of dichloromethane and the solution was cooled to 4° C. 1.2 ml (16.6 mmol) of TFA were then added dropwise and the resulting reaction mixture was kept stirring at ambient temperature for 90 min. It was verified that the reaction was complete by TLC (CH₂Cl₂/MeOH, 80:20, v/v) then the reaction mixture was evaporated to dryness. The oily residue thus obtained was dissolved in distilled water and lyophilized. 199 mg (0.27 mmol) of the coupling product (B-1-C-2) was obtained in the form of a white powder with a quantitative yield. This compound was then used in the following step without supplementary purification.

¹H NMR analysis (300 MHz, CD₃OD): δ=1.26-1.28 (t, 3H, J=3.8 Hz, CH₃(SEt)Cys), 1.40-1.56 (m, 4H, CH₂, β, γ Lys), 1.83-1.90 (m, 2H, CH₂ 8 Lys), 2.67-2.74 (q, 2H, J=7.2 Hz, CH₂(SEt) Cys), 2.93-2.96 (d, 2H, J=9.4 Hz, CH₂β Cys), 3.20-3.28 (m, 2H, CH₂ε Lys), 3.84-3.88 (t, J=6.0 Hz, 1H, (H α Lys), 4.23 (s, 3H, CH Fmoc, CH₂), 4.46-4.48 (d, 2H, J=6.4 Hz, Ch₂ Fmoc), 4.63-4.68 (q, J=4.9 Hz, 1H, CH α Cys), 7.26-7.78 (m, 8H, CH Fmoc), 8.32 (bs, 1H, NH).

¹³C NMR analysis (75.5 MHz, CD₃OD): δ=14.7 (CH₃(SEt) Cys), 22.3 (CH₂γ, Lys) 30.7 (CH₂ δ Lys), 32.2 (CH₂ (SEt) Cys), 33.2 (CH₂β Lys), 39.5 (CH₂β Cys), 41.0 (CH₂ε Lys), 48.2 (CH Fmoc), 53.9 (CH α Cys), 54.2 (CH α Lys), 68.6 (CH₂ Fmoc), 76.5 (CH₂), 121.0 (CH Fmoc), 126.0 (CH Fmoc), 128.2 (CH Fmoc), 129.0 (CH Fmoc), 142.7-174.3 (Cq).

RP-HPLC (system B): t_{R}=16.5 min, 74% purity.

MS (MALDI-TOF, positive mode) m/z=604.70 (M+H)⁺, 626.68 (M+Na)⁺, 642.66 (M+K)⁺, calculated for C₂₈H₃₇N₅O₆S₂ 603.76.

### c) Third Step: Synthesis of the Boc Protected Trifunctional Reagent (22)

78 mg (0.19 mmol) of the precursor (A-1) as prepared previously in example 1 were dissolved in 2 ml of anhydrous CH₃CN. 31 mg (0.23 mmol) of hydroxybenzotriazole monohydrate and 47.3 mg (0.23 mmol) of DCC were then added and the resulting reaction mixture was kept stirring at ambient temperature for 2 hours under an argon atmosphere. Next, 136.4 mg (0.19 mmol) of the compound (B-1-C-2) obtained above in the preceding step were added, then the reaction mixture was kept stirring at ambient temperature for 2 hours under an argon atmosphere. The round-bottomed flask containing the reaction mixture was stored at a temperature of −20° C. overnight. The next day, 32 μl (0.19 mmol) of DIEA were added. The reaction was monitored by RP-HPLC (system B). At the end of 3 hours, a supplementary amount of DCC (12 mg, 58 μmol) and of DIEA (16 μl, 95 μmol) was added. At the end of 90 min, 16 μl (95 μmol) of DIEA were again added. 2 hours later, 16 μl (95 μmol) of DIEA were once again added. The reaction was stopped by addition of 21 μl (360 μmol) of acetic acid and the round-bottomed flask containing the reaction medium was stored at a temperature of −20° C. overnight. After dilution with 2 ml of a CH₃CN/H₂O mixture (2:1, v/v), the expected compound (22) was purified by RP-HPLC (system C, that is to say using a Varian Kromasil® C₁₈ column of 10 μm and of dimensions 21.2×250 mm with, as eluent, a mixture of acetonitrile and of osmosed water by passing 90% of osmosed water for 5 minutes then a linear gradient ranging from 10 to 40% of CH₃CN over 15 minutes, then from 40% to 70% of CH₃CN, with a flow rate of 20.0 ml/min; a double detection UV analysis was carried out at 254 and 305 nm). Two products were obtained and lyophilized in order to result respectively in 38 mg and 66 mg of two different trifunctional reagents (22-1) (22-2) in the form of a white powder with an overall yield of 61%.

Compound (22-1)=expected compound (22): RP-HPLC (system B): t_{R}=20.8 min, 86% purity, 66 mg.

¹H NMR analysis (300 MHz, CDCl₃): δ=1.25-1.30 (t, 3H, J=7.1 Hz, CH₃(SEt)Cys), 1.43 (s, 9H, tBu), 1.52-1.90 (m, 4H, CH₂γ, δ Lys), 2.32 (bs, 2H, CH₂β Lys), 2.64-2.69 (q, 2H, J=7.2 Hz, CH₂(SEt) Cys), 2.97-3.16 (m, 2H, CH₂β Cys), 3.30 (s, 2H, CH₂ unit (A-1)), 3.45-3.65 (m, 11H, CH₂ε Lys+4×CH₂ unit (A-1)), 4.0 (s, 3H, CH₂ unit (A-1)+CH Fmoc), 4.21-4.25 (q, J=6.8 Hz, 1H, CH α Lys), 4.34 (s, 2H, CH₂), 4.44-4.50 (d, 2H, J=10.0 Hz, CH₂ Fmoc), 4.70-4.72 (q, 1H, J=2.3 Hz CH α Cys), 5.23 (bs, 1H, NH), 6.08 (bs, 1H, NH), 6.9 (bs, 1H, NH), 7.26-7.77 (m, 8H, CH Fmoc).

¹³C NMR analysis (75.5 MHz, CDCl₃): δ=14.4 (CH₃(SEt) Cys), 22.6 (CH₂γ, Lys) 26.7 (CH₂ unit (A-1)), 28.5 (tBu), 28.7 (CH₂ δ Lys), 31.7 (CH₂ (SEt) Cys), 32.5 (CH₂ β Lys), 38.6 (CH₂ unit (A-1)), 38.8 (CH₂ unit (A-1)), 39.6 (CH₂ε Lys), 40.4 (CH₂β Cys), 47.0 (CH₂ unit (A-1)), 53.6 (CH α Lys), 68.0 (CH₂ Fmoc), 70.0-71.2 (5×CH₂ unit (A-1)), 77.5 (CH₂ unit (A-1)), 77.8 (CH Fmoc), 79.5 (Cq tBu), 120.2 (CH Fmoc), 125.1 (CH Fmoc), 127.3 (CH Fmoc), 128.1 (CH Fmoc), 141.4-173.0 (Cq).

MS (MALDI-TOF, positive mode) m/z=1016.66 (M+Na)⁺, calculated for C₄₅H₆₇N₇O₁₄S₂.Na 1017.20.

Compound (22-2) (not expected): RP-HPLC (system B): t_{R}=21.6 min, 79% purity, 38 mg.

MS (MALDI-TOF, positive mode) m/z=871.47 (M+Na)⁺, calculated for C₃₉H₅₆N₆O₁₁S₂.Na 871.04.

### d) Fourth Step: Deprotection of the Compound (22-1) and Synthesis of the Trifunctional Reagent of Formula (I-2)

62 mg (0.062 mmol) of the compound (22-1) obtained above in the preceding step were dissolved in 3 ml of dichloromethane. The solution was cooled to 4° C. and 368 μl (4.96 mmol) of TFA were then added dropwise. The resulting reaction mixture was kept stirring at ambient temperature for 1 hour. It was verified that the reaction was complete by RP-HPLC (system B) then the mixture was evaporated to dryness. A minimal amount of osmosed water was then added and the resulting solution was lyophilized in order to result in 83 mg (0.082 mmol) of the compound (22-1) (TFA salt) in deprotected form (white powder).

This compound was then used in the following reactions without supplementary purification.

RP-HPLC (system B): t_{R}=16.5 min, 88% purity.

MS (MALDI-TOF, positive mode) m/z=894.64 (M+H)⁺, calculated for C₄₀H₅₉N₇O₁₂S₂ 894.08.

36 mg (36.7 μmol) of the TFA salt of the deprotected compound (22-1) obtained above were dissolved in 500 μl of anhydrous DMF. 5 μl (36.7 μmol) of TEA and a solution of DSC (24 mg, 91.75 μmol) in 250 μl of anhydrous DMF were then added dropwise and the reaction mixture was kept stirring at ambient temperature for 90 minutes. It was verified that the reaction was complete by RP-HPLC (system B). The mixture was then taken up in around 4 ml of a 0.1% aqueous solution of TFA and purified by RP-HPLC (system D, that is to say by using a Thermo Hypersil GOLD®C₁₈ column of 5 μm and of dimensions 10×250 mm with, as eluent, an acetonitrile/0.1% (v/v, pH 2) aqueous solution of TFA mixture by passing the mixture containing 90% of 0.1% TFA for 5 minutes then a linear gradient ranging from 10 to 40% of CH₃CN over 15 minutes, then 40% to 70% of CH₃CN, with a flow rate of 5.0 ml/min; a double detection UV analysis was carried out at 254 and 305 nm). The fractions containing the product were lyophilized in order to result in 25 mg (24.2 μmol) of the trifunctional reagent of formula (I-2) in the form of a white amorphous powder with a yield of 74%. RP-HPLC (system B): t_{R}=18.5 min, 97% purity.

¹H NMR analysis (300 MHz, CDCl₃): δ=1.24-1.27 (t, 3H, J=7.1 Hz, CH₃(SEt)Cys), 1.32-1.90 (m, 6H, CH₂β, γ, δ Lys), 2.63-2.69 (q, 2H, J=7.1 Hz, CH₂(SEt) Cys), 2.81 (s, 4H, 2×CH₂ succinimidyl), 2.24-3.28 (m, 2H, CH₂β Cys), 3.24-3.69 (m, 11H, 5×CH₂ unit (A-1)+CH₂ε Lys), 4.22-4.27 (t, 1H, CH Fmoc), 4.32 (s, 2H, CH₂), 4.51-4.53 (d, 2H, J=6.4 Hz, CH₂ Fmoc), 4.58-4.66 (q, J=7.5 Hz, 1H, CH α Lys), 4.70-4.76 (q, 1H, J=6.0 Hz CH α Cys), 6.17 (bs, 1H, NH), 6.83 (s, 1H, NH), 7.27-7.83 (m, 8H, CH Fmoc), 9.2 (bs, 1H, NH).

MS (MALDI-TOF, positive mode) m/z=1035.17 (M+H)⁺, 1057.71 (M+Na)⁺, 1073.68 (M+K)⁺, calculated for C₄₅H₆₂N₈O₁₆S₂ 1035.69 (M+H)⁺.

The trifunctional reagent of formula (I-2) can then be functionalized by any fluorescent ligand in order to result in the corresponding fluorescent trifunctional reagent.

1. A pseudopeptide trifunctional reagent, comprising:
(a) a unit A comprising a hydrophilic chain of pseudo-polyethylene glycol interrupted by at least one amide functional group and having two ends E1 and E2, said end E1 being free and comprising an amino group, activated carbamates or activated esters, or mixtures thereof, and said end E2 comprising a terminal carbon atom bearing a carbonyl functional group, said carbon atom being engaged in an amide (—C(O)—NH—) functional group formed with the nitrogen atom of an α-amine functional group borne by the unit B;
(b) a unit B comprising at least one amino acid selected from the group of α-amino acids consisting of the L or D series, and racemic mixtures thereof, said amino acid having on its side chain at least one oxyamine functional group protected by a protecting group or at least one masked aldehydic functional group; and
(c) a unit C comprising at least one amino acid selected from the group of α-amino acids consisting of the L or D series, and racemic mixtures thereof, said amino acid having on its side chain at least one thiol, maleimide, iodoacetyl, azide, true alkyne, phosphane or cyclooctyne unit;
said units B and C being linked together via an amide functional group formed between the carbon atom bearing the carbonyl functional group of the α-amino acid of the unit B and the nitrogen atom of the α-amine functional group of the α-amino acid of the unit C.

2. The trifunctional reagent as claimed in claim 1, wherein the pseudo-polyethylene glycol chain of the unit A is represented by formula (A-I) below: 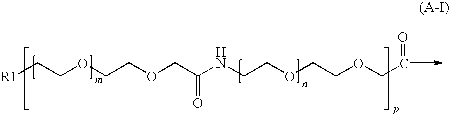
in which:
R₁ represents a primary amine, an activated carbamate unit or an activated ester unit,
m and n, which are identical or different, are integers between 2 and 10 inclusive,
p is an integer between 1 and 10 inclusive,
the arrow represents the covalent bond connecting the amide functional group of the end E2 of the pseudo-polyethylene glycol chain to the unit B or C.

3. The trifunctional reagent as claimed in claim 2, wherein the pseudo-polyethylene glycol chain is selected from the group consisting of pseudo-polyethylene glycol chains of formula (A-I) in which m=n=2 and p=1.

4. The trifunctional reagent as claimed in claim 1, wherein the activated carbamate groups are selected from the group consisting of N-hydroxysuccinimidyl carbamate, sulfo-N-hydroxysuccinimidyl carbamate, N-hydroxyphthalimidyl carbamate, N-hydroxypiperidyl carbamate, p-nitrophenyl carbamate and pentafluorophenyl carbamate.

5. The trifunctional reagent as claimed in claim 4, wherein the activated carbamate group is N-hydroxysuccinimidyl carbamate.

6. The trifunctional reagent as claimed in claim 1, wherein the activated ester groups are selected from the group consisting of N-hydroxy-succinimidyl ester, sulfo-N-hydroxysuccinimidyl ester, cyanomethyl ester, N-hydroxyphthalimidyl ester, N-hydroxypiperidyl ester, p-nitrophenyl ester, pentafluorophenyl ester, a benzotriazole ester, a hydroxybenzotriazole ester and a hydroxyazabenzotriazole ester.

7. The trifunctional reagent as claimed in claim 6, wherein the activated ester group is N-hydroxysuccinimidyl ester.

8. The trifunctional reagent as claimed in claim 1, wherein the α-amino acids of the unit B are selected from the group consisting of lysine, homolysine, ornithine, 2,4-diaminobutanoic acid and 2,3-diaminopropanoic acid.

9. The trifunctional reagent as claimed in claim 8, wherein the α-amino acid of the unit B is lysine.

10. The trifunctional reagent as claimed in claim 1, wherein the α-amino acids for the unit C are selected from the group consisting of lysine, cysteine, homolysine, ornithine, 2,4-diaminobutanoic acid, 2,3-diaminopropanoic acid, aminomercaptoacetic acid, homocysteine, 5-mercaptonorvaline, 6-mercaptonorleucine, 2-amino-7-mercaptoheptanoic acid and 2-amino-8-mercaptooctanoic acid.

11. The trifunctional reagent as claimed in claim 10, wherein the α-amino acid of the unit C is lysine or cysteine.

12. The trifunctional reagent as claimed in claim 1, wherein the protecting groups of the oxyamine functional group of the side chain of the α-amino acid constituting the unit B are selected from the group consisting of 9-fluorenylmethoxycarbonyl, benzyloxycarbonyl, allyloxycarbonyl, trichloroethoxycarbonyl, trimethylsilylethoxycarbonyl, pyridyl-dithio-ethoxy-carbonyl, 2-(2-nitrophenyl)propyloxycarbonyl, azomethyloxycarbonyl, 2-(2-nitro-phenyl)propyl-oxycarbonyl, azomethyloxycarbonyl, 2-(trimethyl-silyl)ethanesulfonyl and a phthalimide.

13. The trifunctional reagent as claimed in claim 1, represented by formula (I) below: 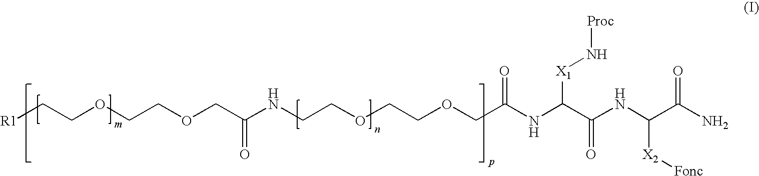
in which:
R₁, m, n and p have the same meanings as those defined in claim 2 or 3 for the unit of formula (A-I),
X₁ and X₂, which are identical or different, represent, together with the carbon atom to which they are attached, the hydrocarbon-based chain of an α-amino acid,
Proc is a protecting group selected from the group consisting of 9-fluorenylmethoxycarbonyl, benzyloxycarbonyl, allyloxycarbonyl, trichloroethoxycarbonyl, trimethyl-silylethoxycarbonyl, pyridyldithioethoxy-carbonyl, 2-(2-nitrophenyl)propyloxycarbonyl, azomethyloxycarbonyl, 2-(trimethyl-silyl)ethane-sulfonyl and phthalimide;
Fonc is a thiol, maleimide, iodoacetyl, azide, true alkyne, phosphane or cyclooctyne unit.

14. The trifunctional reagent as claimed in claim 13, wherein X₁ is an n-butyl chain.

15. The trifunctional reagent as claimed in claim 13 wherein X₂ is an ethyl or n-butyl chain.

16. The reagent as claimed in claim 13, wherein the compounds of formula (I) are selected from the group consisting of formula (I-1) to (I-6):

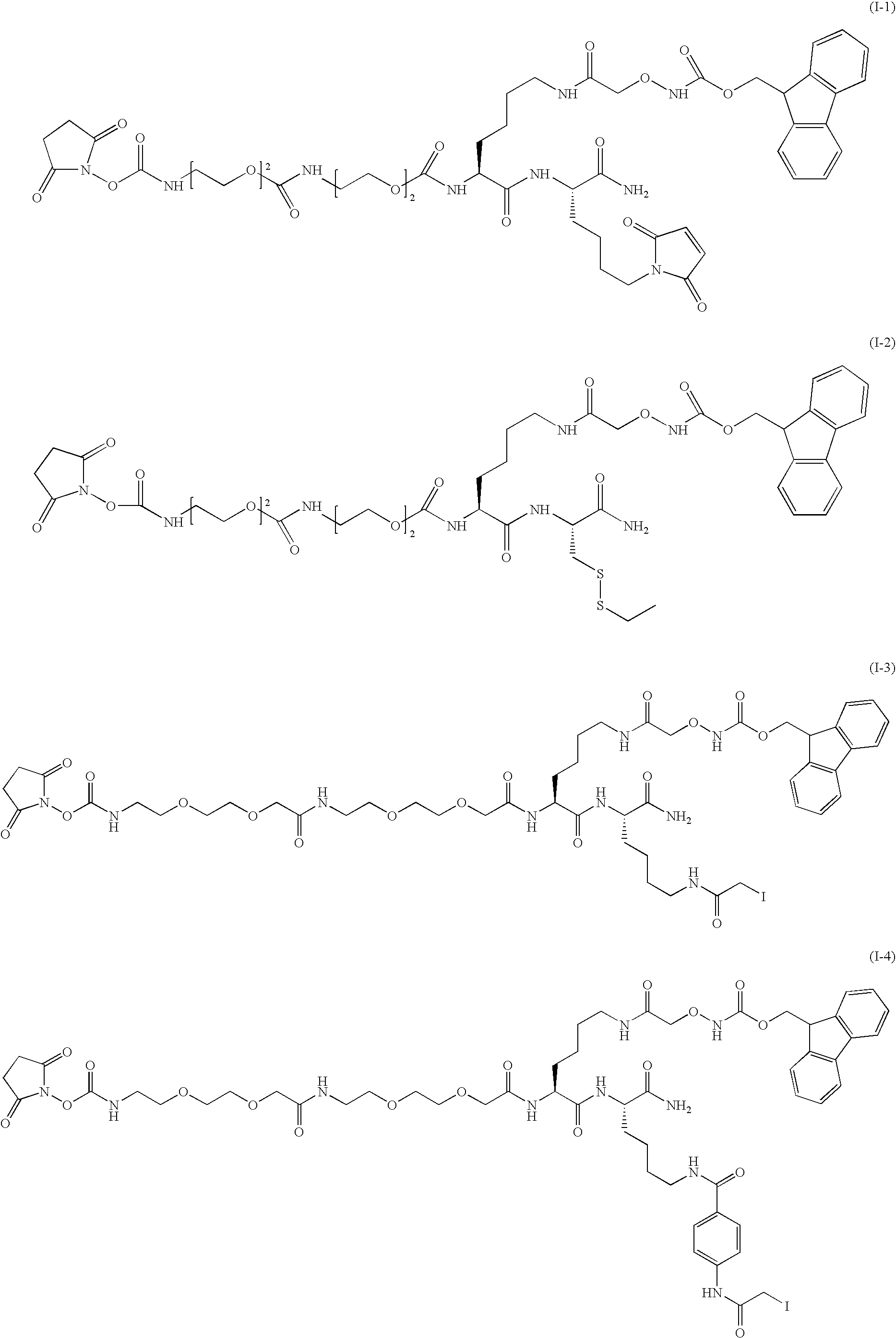

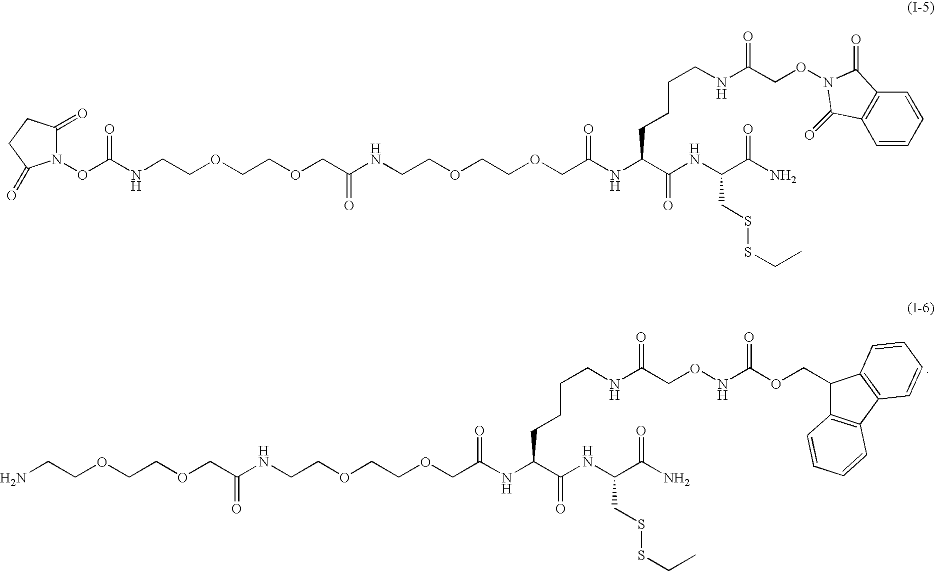

17. (canceled)

18. A bioconjugate, a comprising trifunctional reagent as defined in claim 1, in which the primary amine, activated carbamate or activated ester unit present at the free end of the pseudo-polyethylene glycol chain of the unit A or the oxyamine or aldehydic functional group borne by the unit B after its deprotection, or both, respectively its demasking, is functionalized by a biological molecule of interest.

19. The bioconjugate as claimed in claim 18, wherein the biological molecule of interest is selected from the group consisting of antibodies, molecules of nucleic acids and their analogs, polysaccharides, proteins, peptides, radionuclides, toxins, enzyme inhibitors and haptens.

20. The bioconjugate as claimed in claim 18, wherein said bioconjugate is at least one selected from the group consisting of
i) a bioconjugate comprising a trifunctional reagent in which only the primary amine functional group or only the activated carbamate or activated ester unit present at the free end of the pseudo-polyethylene glycol chain of the unit A is functionalized by a biological molecule,
ii) a bioconjugate comprising a trifunctional reagent in which only the deprotected oxyamine or demasked aldehydic functional group of the unit B is functionalized by a biological molecule, and
iii) a bioconjugate comprising two biological molecules, comprising a trifunctional reagent in which the primary amine functional group or activated carbamate or activated ester unit present at the free end of the pseudo-polyethylene glycol chain of the unit A and the deprotected oxyamine or demasked aldehydic functional group of the unit B are each functionalized by a biological molecule, said molecules being identical to or different from one another.

21. (canceled)

22. A luminescent trifunctional reagent, comprising a trifunctional reagent as defined in claim 1, further comprising a single luminescent group (L) attached to the unit B via the deprotected oxyamine functional group or the demasked aldehydic functional group or to the unit C via the thiol functional group or maleimide, iodoacetyl, true alkyne, phosphane, cyclooctyne or else azide units.

23. A luminescent trifunctional reagent, comprising a trifunctional reagent as defined in claim 1, further comprising two luminescent groups, one being attached to the unit B via the deprotected oxyamine functional group or the demasked aldehydic functional group and the other being attached to the unit C via the thiol functional group or the maleimide, iodoacetyl, true alkyne, phosphane, cyclooctyne or else azide units.

24. The luminescent trifunctional reagent as claimed in claim 22, wherein the luminescent group is at least one selected from the group consisting of fluorophores comprising polymethine chains; fluorescent cyanines; fluorescein and derivatives thereof; rhodamine and derivatives thereof; the water-soluble derivatives of rhodamine in the form of ester of N-hydroxysuccinimide; rhodols and derivatives thereof; coumarin derivatives; fluorescent dyes comprising reactive amines; dipyrromethene boron difluorides; fluorophores derived from pyrene; diazo derivatives; dansyl derivatives; eosin; erythrosine and sulforhodamine derivatives.

25. (canceled)

26. An energy transfer cassette, comprising a trifunctional reagent as defined in claim 1, said reagent further comprising a luminescent group (L) and an acceptor compound (Q) that accepts the luminescence from the luminescent group, L and Q being respectively and indifferently attached to said trifunctional reagent via the deprotected oxyamine or demasked aldehydic functional group of the unit B and the thiol functional group or the maleimide, iodoacetyl, true alkyne, phosphane, cyclooctyne or else azide units of the unit C.

27. The cassette as claimed in claim 26, wherein the luminescent group (L) is selected from the group consisting of fluorophores comprising polymethine chains; fluorescent cyanines; fluorescein and derivatives thereof; rhodamine and derivatives thereof; the water-soluble derivatives of rhodamine in the form of ester of N-hydroxysuccinimide; rhodols and derivatives thereof; coumarin derivatives; fluorescent dyes comprising reactive amines; dipyrromethene boron difluorides; fluorophores derived from pyrene; diazo derivatives; dansyl derivatives; eosin; erythrosine and sulforhodamine derivatives.

28. The cassette as claimed in claim 25, wherein the acceptor compound (Q) is selected from the group consisting of luminescent groups (L) listed in claim 27, non-fluorescent molecules of the family of azo dyes, gold particles, diazo dyes, malachite green and the acceptor compounds of the family of cyanines.

29. The cassette as claimed in claim 25, wherein the luminescent group (L) and the acceptor compound (Q) are selected from the following (L/Q) pairs: Cy3/Cy5, Cy5/Cy7, Cy5/Alexa Fluor® 750, Cy3/Cy5Q, Cy3/QSY® 7, Cy3/QSY® 9, Cy5/Cy7Q, Cy5/QSY® 21, Cy5/Cy5, Cy5.5/Cy5.5, Cy7/Cy7, R6)/Cy5, R6G/Alexa Fluor® 647, R6G/QSY® 21, Alexa Fluor® 532/Cy5, Alexa Fluor® 532/Alexa Fluor® 647, Alexa Fluor® 532/QSY® 21, Alexa Fluor® 555/Cy5, Alexa Fluor® 555/Alexa Fluor® 647, Alexa Fluor® 555/QSY® 21.

30. A method of preparing a mixed bioconjugate, comprising mixing a trifunctional reagent as defined in claim 1, at least one biological molecule, and at least one luminescent group.

31. A mixed bioconjugate, comprising a trifunctional reagent as defined in claim 1, wherein said trifunctional reagent is, attached to at least one biological molecule and at least one luminescent group.

32. The mixed bioconjugate as claimed in claim 31, further comprising an acceptor compound (Q) that accepts the luminescence from the luminescent group.

33. The mixed bioconjugate as claimed in claim 31 wherein said mixed bioconjugate is selected from the group of trifunctional reagents consisting of:
i) one or two biological molecules and a luminescent group; or
ii) one biological molecule and two luminescent groups;
iii) one biological molecule, one luminescent group, and one acceptor compound (Q) that accepts the luminescence from the luminescent group;
said biological molecules, said luminescent groups and said acceptor compound (Q) being attached to the functional reagent at the terminal ends of the units A, B and C.

34. A method of functionalizing a solid support, comprising functionalizing a solid support with a trifunctional reagent as defined in claim 1, wherein the solid support comprises at least one surface comprising one or more carboxyl-containing groups or one or more oxyamine functional groups, and wherein the oxyamine or respectively aldehydic functional group of the unit B of said trifunctional reagent is free.

35. A solid support, comprising at least one surface that is functionalized, covalently, by one or more trifunctional reagents, or by one or more bioconjugates as defined in claim 1, or both, said trifunctional reagents and bioconjugates optionally being modified by a luminescent group at the unit C.

36. The solid support as claimed in claim 35, comprising at least one surface functionalized by at least one bioconjugate to produce a biochip or a biosensor.

37. A method of detecting a molecule of interest comprising detecting a molecule of interest with a support as defined in claim 35 or 36.

38. (canceled)

39. A probe for functional proteomics, comprising a trifunctional reagent as defined in claim 1, said reagent comprising:
a group that enables the detection (or visualization) or the purification of a target protein (reporter tag), or both,
a unit recognized by said target protein (recognition unit), and
a reactive group that enables a covalent bond to be established with the active site of the target protein (reactive group).

40. The probe as claimed in claim 39, wherein the target protein is an enzyme.
